(19) United States
(12) Patent Application Publication
(10) Pub. No.: US 2010/0003224 A1
(43) Pub. Date: Jan. 7, 2010

(54) Combination Therapy

(75) Inventors: Gary J. Bridger, Bellingham, WA (US); Louis M. Pelus, Carmel, IN (US)

Correspondence Address: GENZYME CORPORATION;LEGAL DEPARTMENT, 15 PLEASANT ST CONNECTOR, FRAMINGHAM, MA, 01701-9322, US

(73) Assignee: GENZYME CORPORATION, Cambridge, MA (US)

(21) Appl. No.: 12/375,987

(22) PCT Filed: Aug. 2, 2007
(86) PCT No.: PCT/US07/75064

§ 371 Date: Feb. 2, 2009

Related U.S. Application Data
(60) Provisional application No. 60/835,290, filed on Aug. 2, 2006.

(51) Int. Cl. A61K 35/14; A61K 38/16; A61K 31/395; A61K 31/40

(52) U.S. Cl. 424/93.7; 514/12; 514/183; 514/423

(57) ABSTRACT

Methods to mobilize progenitor and/or stem cells from the bone marrow to the bloodstream by administering a combination of at least one CXCR4 inhibitor and at least one VLA-4 inhibitor are described. The combinations may also be used to treat multiple myeloma.

### RELATED APPLICATION

This application claims benefit of U.S. provisional application Ser. No. 60/835,290 filed 2 Aug. 2006 which is incorporated herein by reference in its entirety.

### TECHNICAL FIELD

The invention is in the field of therapeutics and medicinal chemistry. More particularly, the invention concerns methods to rapidly mobilize progenitor/stem cells, including pre-cancerous progenitor and/or stem cells into the blood stream using combination therapy.

### BACKGROUND ART

Peripheral Blood Stem Cell Transplant (PBSCT) is a new technique in which progenitor and/or stem cells are obtained from a patient's blood and used to restore the immune system of patients (including, in some instances, the donor) who have had chemotherapy and/or radiation therapy. To obtain the stem cells, these cells must be mobilized or moved from the bone marrow into the peripheral blood. The strongest predictor of success in such transplantation, measured by the rapid and durable recovery of a patient's immune system, is the number of stem cells available for transplantation. Stem cell transplantation can be characterized as either allogeneic, where cells are transplanted from a healthy donor, usually a sibling, or as autologous, where cells are collected from the patient and reinfused after chemotherapy.

The current strategies of mobilizing bone marrow progenitor and/or stem cells into the blood stream employ growth factors such G-CSF (Neupogen®). G-CSF can be used alone or combined with chemotherapeutic drugs such as Cytoxan. In both cases, mobilization for progenitor and/or stem cells requires approximately 5-10 days of G-CSF treatment and is associated with significant side-effects such as bone pain or febrile neutropenia.

Stem cell collection, a process called apheresis, can take up to 4 to 5 hours. Using intravenous tubes the patient's blood is continually circulated through an apheresis machine and back into the patient. The apheresis machine separates different types of blood and immune cells. A patient may require multiple apheresis sessions before a sufficient amount of stem cells are collected for a stem cell transplant. When G-CSF is used to mobilize, administration of G-CSF is continued on apheresis days. Once the target number of stem cells has been collected, they are stored until used for transplantation.

In some embodiments, the donor/patient receives chemotherapy to treat cancer. This treatment not only destroys the cancer but also seriously damages the immune system. Following chemotherapy, and once the patient has been stabilized, the stored stem cells can be transplanted back into the patient, through an intravenous infusion. Patients are given antibiotics and blood transfusions to prevent infection while their immune systems are recovering. Once in the bloodstream the stem cells migrate back into the bone marrow. Over a period of 11-30 days, these stem cells will increase in number and develop into different types of cells including platelets and immune cells such as neutrophils.

Other factors or agents have been reported to increase circulating white blood cells and progenitor cells in both human and animal subjects. These agents include AMD3100, granulocyte-macrophage colony stimulating factor (GM-CSF), Interleukin-1 (IL-1), Interleukin-3 (IL-3), Interleukin-8 (IL-8), PIXY-321 (GM-CSF/IL-3 fusion protein), macrophage inflammatory protein, stem cell factor (SCF), thrombopoietin, flt3, myelopoietin, anti-VLA-4 antibody, anti-VCAM-1 and growth related oncogene (GRO). These may be used as single agents or in combination (Dale, D., et al., Am. J. of Hematol. (1998) 57:7-15; Rosenfeld, C., et al., Bone Marrow Transplantation (1997) 17:179-183; Pruijt, J., et al., Cur. Op. in Hematol. (1999) 6:152-158; Broxmeyer, H., et al., Exp. Hematol. (1995) 23:335-340; Broxmeyer, et al., Blood Cells, Molecules and Diseases (1998) 24:14-30; Glaspy, J., et al., Cancer Chemother. Pharmacol. (1996) 38(suppl):S53-S57; Vadhan-Raj, S., et al., Ann. Intern. Med. (1997) 126:673-681; King, A., et al., Blood (2001) 97:1534-1542; Glaspy, J., et al., Blood (1997) 90:2939-2951; and Papayannopoulou, T., et al., Proc. Natl. Acad. Sci. USA (1995) 92:9647-9651).

Very late antigen-4 (VLA-4), also known as α₄β₁ is a heterodimeric cell surface molecule found on all hemopoietic mononuclear cells and eosinophils. The natural ligands of VLA-4 are vascular cell adhesion molecule (VCAM-1) and alternatively spliced connecting segment (CS-1) of fibronectin. VCAM-1 is found constitutively on endothelial cells and smooth muscle cells. The expression of VCAM-1 on other cell types is upregulated and induced by cytokines, such as tumor necrosis factor-α, interleukin-1 (IL-1), and interleukin-4 (IL-4). The constitutive or nonactivated form of VLA-4 requires activation to bind to VCAM-1. Activation of VLA-4 is achieved by cytokines, such as IL-1 and IL-4, as well as certain stimulating antibodies, such as anti-CD3 and CD28 Ab, resulting in high-affinity binding and promoting trans-endothelial diapedesis. VLA-4 receptor is primarily responsible for mononuclear cell trafficking. VLA-4 antagonists have been proposed to be effective therapeutically in diseases where cell accumulation or recruitment is the causative element. Studies have shown that anti-VLA-4 antibody and VLA-4 antagonists can inhibit cellular accumulation in animal models of allergic encephalitis, non-obese diabetes, asthma, and rheumatoid arthritis. Studies have shown the administration of antibodies to VLA-4 mobilized hematopoietic progenitors into the circulating blood mice and primates, as a single agent or in combination with growth factors such as G-CSF. Recently, a humanized anti-VLA-4 antibody (Tysabri®, natalizumab) was approved for the treatment of multiple sclerosis (Miller, D. H., et al., N. Engl. J. Med, (2003) 348:15-21).

The chemokine receptor CXCR4 and its natural ligand stromal cell derived factor-1 (SDF-1) appear to be important in the process of development and maturation of blood cells wherein mature blood cells are derived from hematopoietic precursor cells (progenitor) cells and stem cells present in specific hematopoietic tissues including bone marrow (for reviews see Maekawa, T., et al., Internal Med. (2000) 39:90-100; Nagasawa, T., et al., Int. J. Hematol. (2000) 72:408-411). This is demonstrated by reports that CXCR4 or SDF-1 knock-out mice exhibit hematopoietic defects (Ma, Q., et al., Proc. Natl. Acad. Sci. USA (1998) 95:9448-9453; Tachibana, K., et al., Nature (1998) 393:591-594; Zou, Y-R., et al., Nature (1998) 393:595-599). It is also known that CD34+ progenitor cells express CXCR4 and require SDF-1 produced by bone marrow stromal cells for chemoattraction and engraftment (Peled, A., et al., Science (1999) 283:845-848) and that in vitro, SDF-1 is chemotactic for both CD34+ cells (Aiuti, A., et al., J. Exp. Med. (1997) 185:111-120; Viardot, A., et al., Ann. Hematol. (1998) 77:194-197) and for progenitor/stem cells (Jo, D-Y., et al., J. Clin. Invest. (2000) 105:101-111). SDF-1 is also an important chemoattractant, signaling via the CXCR4 receptor, for several other more committed progenitors and mature blood cells including T-lymphocytes and monocytes (Bleul, C., et al., J. Exp. Med. (1996) 184:1101-1109), pro- and pre-B lymphocytes (Fedyk, E. R., et al., J. Leukoc. Biol. (1999) 66:667-673; Ma, Q., et al., Immunity (1999) 10:463-471) and megakaryocytes (Hodohara, K., et al., Blood (2000) 95:769-775; Riviere, C., et al., Blood (1999) 95:1511-1523; Majka, M., et al., Blood (2000) 96:4142-4151; Gear, A., et al., Blood (2001) 97:937-945; Abi-Younes, S., et al., Circ. Res. (2000) 86:131-138).

Recent studies suggest cross talk between SDF-1 signaling and VLA-4 mediated adhesion of cells within the bone marrow, where evidence indicates that an increase in VLA-4 adhesive activity by SDF-1 could represent an important initial step in the homing of CD34+ progenitor cells to the bone marrow (Hidalgo, A. et al., Ex. Hematol (2001) 29:345-355; Peled, A., et al., J Clin Invest (1999) 104:1199-1211).

The CD34+ population is the component thought to be primarily responsible for the improved recovery time after chemotherapy and the cells most likely responsible for long-term engraftment and restoration of hematopoiesis (Croop, J. M., et al., Bone Marrow Transplantation (2000) 26:1271-1279). The mechanism by which CD34+ cells re-engraft may be due to the chemotactic effects of SDF-1 on CXCR4 expressing cells (Voermans, C., Blood (2001) 97:799-804; Ponomaryov, T., et al., J. Clin. Invest. (2000) 106:1331-1339). Furthermore, studies also show that adult hematopoietic stem cells are capable of restoring damaged cardiac tissue in mice (Jackson, K., et al., J. Clin. Invest. (2001) 107:1395-1402; Kocher, A., et al., Nature Med. (2001) 7:430-436).

Thus, the role of the CXCR4 receptor in managing cell positioning and differentiation has assumed considerable significance. The compound AMD3100, which is 1,1[1,4-phenylene-bis(methylene)]-bis-1,4,8,11-tetraazacyclotetradecane, is a known CXCR4 antagonist which itself mobilizes progenitor cells (see, for example, Hubel, K., et al., Supportive Cancer Therapy (2004) 1:165-172, citing De Clercq, E., et al., Nat. Rev. Drug Discov. (2003) 2:581-587. In addition, PCT publication WO 00/45814 discloses that various cyclic polyamine compounds, including AMD3100, elevate white blood cell counts. WO 03/011277 further shows that such compounds, including AMD3100, mobilize progenitor/stem cells to permit their harvest and to rebuild damaged cardiac tissue. A combination of AMD3100 with various other factors, including GM-CSF, IL-1, IL-3, IL-8, PIXY-321 macrophage inflammatory protein, skin cell factor, thrombopoietin, growth-related oncogene or chemotherapy, or additional active ingredients generally, such as antibiotics, vitamins, herbal extracts, anti-inflammatories, glucose, anti-pyretics, analgesics is also mentioned. AMD3100 was shown to have protective effects in collagen-induced arthritis models in mice (Matthys, P., et al., J. Immunol. (2001) 167:4686-4692). WO 06/020891 describes the use of the combination of CXCR4 antagonist with a GROβ protein for stem cell mobilization.

WO 06/023396 teaches that inhibiting the interaction between VLA-4 and its ligands using anti-α4 antibodies or blocking peptides is therapeutically efficacious in several animal models of disease including asthma, multiple sclerosis, inflammatory bowel disease, and rheumatoid arthritis. Humanized monoclonal antibody against α4, natalizumab (Antegren®, Elan/Biogen), has demonstrated efficacy in the treatment of multiple sclerosis. WO 06/036371 describes combination therapy for the treatment of immune or inflammatory disorder such as multiple sclerosis, with an anti-VLA-4 antibody and a second biologic agent.

While the majority of patients who serve as stem cell donors provide an adequate quantity of cells, a significant number of patients fail to collect the minimum number of stem cells in order to proceed to transplantation. It has been found that between 60-75% of patients do not receive an optimal number of cells upon transplant (Center for International Blood and Marrow Transplant Research (CIBMTR) Registry Data 1998-2002). As a result, these patients have to go through additional stem cell collection sessions to achieve a sufficient number of stem cells. Many of these patients are at a greater risk for serious infections that require antibiotic treatments, blood transfusions and extended hospitalization. In the worst case, some patient's immune systems do not recover and they die of infection.

It has now been found that the combination of a CXCR4 antagonist with a VLA-4 antagonist is particularly effective in mobilizing stem and/or progenitor cells. The mobilization of cells from bone marrow is also relevant in the context of chemotherapy directed to hematopoietic-based malignancies. In particular, chemotherapy or radiation therapy of leukemia may be less effective if the leukemic or pre-leukemic cells are retained in or attracted to the bone marrow rather than remaining available in the circulation where they are more susceptible to treatment.

Within the microenvironment of the bone marrow, SDF-1 acts as a potent chemoattractant for immature and mature hematopoietic cells, and thus expression of CXCR4 on leukemic progenitor cells may contribute to homing them to the bone marrow microenvironment. Elevated CXCR4 levels are detected on leukemic cells from patients with B chronic lymphocytic leukemia (B-CLL) (Mohle, R., et al., Leukemia (1999) 13:1954-1959). However, enhanced levels are not detected on leukemic cells from patients with T-ALL or leukemic cells from patients with AML (Mohle, et al., supra; Voermans, C., et al., Leukemia (2002) 16:650-657; Bradstock, K. F., et al., Leukemia (2000) 14:882-888; Dialynas, D. P., et al., Stem Cells (2001) 19:443-452; Shen, W., et al., Exp. Hematol. (2001) 29:1439-1447). It further appears that autocrine secretion of SDF-1 by blood-derived adherent nurse-like cells in chronic lymphocytic leukemia (CLL) protects leukemic B cells from spontaneous apoptosis (Burger, J. A., et al., Blood (2000) 96:2655-2663). Expression levels of CXCR4 vary among various types of AML (Rombouts, E. J., et al., Blood (2004) 104:550-557; Fukuda, S., et al., Blood (2005) 105:3117-3126). CXCR4 is also reported to mediate homing and engraftment of pre-B-ALL and AML cells to bone marrow, although other factors may be involved (Shen, et al., supra; Tavor, S., et al., Cancer Res. (2004) 64:2817-2824). These studies suggest that SDF-1/CXCR4 interactions are involved in the microenvironmental regulation of leukemic cells where such interaction may play a role in the resistance of residual, post-chemotherapy AML exposure to additional chemotherapeutic agents. In addition, Papayannopoulou, Blood (2004) 103:1580-1585 describe the ability of either a CXCR4 antagonist or GROβ, which is a CXCR2 agonist to elevate progenitor and/or stem cell populations. Combinations of G-CSF with GROβ/CXCL2 and GROβ₁/CXCL2_{δ4} as mobilizing hematopoietic stem and progenitor cells is described by Pelus, L. M., et al., Blood (2004) 103:110-119.

There is a need to provide such treatment of mobilizing pre-cancerous or cancerous cells out from the bone marrow and into the peripheral blood system, where these cells can be exposed to chemotherapeutic agents. The current invention addresses such need where this invention uses at least one inhibitor of the CXCR4 receptor in combination with at least one VLA-4 inhibitor to potentiate the effects of standard chemotherapeutic agents. The present invention provides combinations for the treatment of patients by the rapid movement of leukemic stem cells including, leukemic cells and their precursors, out from the microenvironment of the bone marrow and into circulating blood prior to, or during, or after the treatment using chemotherapy.

It was recently shown, in an in vitro context, that AMD 3100 blocked SDF-1 induced chemotaxis of pre-B-ALL cells into bone marrow stroma layers, and enhanced the cytotoxic and antiproliferative effects of vincristine and dexamethasone (Juarez, J., et al., Leukemia (2003) 17:1294-1300).

The combination of the invention may be used to treat subject that may or may not require transplantation, and for those requiring transplantation may be used in an allogeneic or autologous or tandem transplantation.

Multiple myeloma (MM) is a B-cell malignancy characterized by the accumulation of plasma cells in the bone marrow and accompanying osteoclastic bone destruction with severe pain. Monoclonal immunoglobulin-secreting myeloma plasma cells express VLA-4 and marrow stromal cells constitutively express VCAM-1. Studies have been performed to determine the therapeutic benefit of anti-VLA-4 antibodies in mouse models of MM in combination with melphalan (Mori, Y., et al., Blood (2004) 104:2149-2154); or as a single agent (Olson, D. L., et al., Mol Cancer Ther (2005) 4:91-99). In both studies, treatment with antibodies to VLA-4, after the myeloma cells have homed to the marrow, produced reductions in circulating levels of IgG2b, the percentage of IgG2b positive myeloma cells in the circulating blood and tumor cell burden in the bone marrow and spleen. Osteoclastic bone destruction was also suppressed leading to an improvement in survival of myeloma-bearing mice. While these effects were shown in the absence of melphalan in the Olson study, melphalan was required to obtain these results by Mori. However, a prophylactic effect of the antibodies alone was shown by Mori.

SDF-1 has also been implicated in the recruitment and activation of osteoclast precursors to sites within the bone marrow in subjects with MM. MM plasma cells are reported to produce significant levels of SDF-1 and MM patients exhibit elevated levels of plasma SDF-1 compared to age-matched subjects. The CXCR4 antagonist T-140 blocked osteoclast formation in vitro and therefore disruption of SDF-1/CXCR4 was suggested as a potential treatment for MM-induced osteolysis (Zannettino, A. C., et al., Cancer Res. (2005) 65:1700-1709). Recently, it has been demonstrated that SDF-1 rapidly and transiently up-regulated VLA-4 mediated myeloma cell adhesion to CS-1/fibronectin and VCAM-1 (Sanz-Rodriguez, F., et al., Blood (2001) 97:346-351; Parmo-Cabanas, M., et al., Exp Cell Res (2004) 294:571-580). In another study, mobilization of myeloma patients with GM-CSF and cyclophosphamide caused a decrease in the plasma levels of SDF-1 and a decrease in the levels of CXCR4 and VLA-4 on myeloma plasma cells (Gazitt, Y., et al., Stem Cells (2004) 22:65-73).

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are incorporated in their entirety by reference herein.

### DISCLOSURE OF THE INVENTION

The invention, in one aspect, is directed to methods of treating animal subjects, in particular, veterinary and human subjects, to enhance the number of progenitor cells and/or stem cells available for harvest. The progenitor and/or stem cells may then be harvested and used in cell transplantation. The methods of the invention employ inhibitors of the CXCR4 receptor such as certain polyamines described below in combination with one or more VLA-4 antagonists. The methods are useful in the context of stem cell transplantation, tissue repair, and in situations where direct in vivo stimulation of hematopoiesis is desirable.

In one aspect, therefore, the invention is directed to a method to elevate the number of circulating progenitor cells and/or stem cells, in a subject, which method comprises administering to said subject an amount of at least one compound that inhibits the CXCR4 receptor, such as that of formula (1) shown below, in combination with at least one VLA-4 antagonist. Surprisingly, the combination of a CXCR4 antagonist, along with the VLA-4 antagonist, is able to rapidly mobilize progenitor and stem cells in a short time.

This is particularly advantageous in the context of providing progenitor and/or stem cells for harvest. The harvested cells may be used in allogeneic or autologous transplantations. The mobilized stem cells may also be circulated to tissues in need of repair in the subject administered the combination. Thus, repair of myocardial tissue may be enhanced in a subject by administration of this combination. In this embodiment, progenitor/stem cells are mobilized from the bone marrow and circulated in vivo for myocardial repair.

In another aspect, the invention is directed to a method to treat a subject afflicted with a hematopoietic malignancy, such as a lymphoma, myeloma or leukemia by mobilizing the malignant cells from the bone marrow into the circulation using a combination of at least one CXCR4 inhibitor with at least one VLA-4 inhibitor or with at least one CXCR2 agonist. The chemotherapeutic methods may be employed prior to, during, or subsequent to the administration of the foregoing combination.

In yet another aspect, the invention is directed to combinations of VLA-4 antagonists and CXCR4 antagonists to augment the effectiveness of chemotherapy in subjects with multiple myeloma and other B-cell malignancies or to treat subjects with these conditions. In this embodiment, combinations of CXCR4 and VLA-4 antagonists can be used for the treatment for multiple myeloma by disruption of the myeloma cell environment in the bone marrow and inhibition of osteoclastic bone destruction including during chemotherapy.

In additional aspects, the invention is directed to pharmaceutical compositions containing at least one CXCR4 inhibitor, such as a compound of formula (1) and at least one VLA-4 antagonist for use in effecting an elevation of progenitor cells and/or stem cells in the circulation of animal subjects, for use in enhancing sensitivity to chemotherapeutic methods or to radiation therapy, and for use in treating multiple myeloma.

### MODES OF CARRYING OUT THE INVENTION

One aspect of the invention relates to the combination of at least one CXCR4 antagonist with at least one VLA-4 antagonist to mobilize progenitor and/or stem cells from bone marrow to the circulation. The combination is able to accomplish this stimulation in a much shorter time than either component alone and in a much shorter time than previously disclosed agents or combinations. The progenitor/stem cells are mobilized more quickly, in higher numbers and over a more prolonged period than when either agent is administered alone. Mobilization of stem cells and/or progenitor cells is useful in a number of contexts, as further described below.

The same combination is also used to mobilize pre-malignant or malignant cells from the bone marrow into the circulation to expose them more effectively to chemotherapy or radiotherapy. In addition, combinations of CXCR4 antagonists and CXCR2 agonists may be used for this purpose. The combination of at least one CXCR4 antagonist and at least one VLA-4 antagonist may also be used to treat subjects for multiple myeloma resulting in a decrease in burden of tumor cells in bone and bone marrow.

As used herein, the term “progenitor cells” refers to cells that, in response to certain stimuli, can form differentiated hematopoietic or myeloid cells. The presence of progenitor cells can be assessed by the ability of the cells in a sample to form colony-forming units of various types, including, for example, CFU-GM (colony-forming units, granulocyte-macrophage); CFU-GEMM (colony-forming units, multipotential); BFU-E (burst-forming units, erythroid); HPP-CFC (high proliferative potential colony-forming cells); or other types of differentiated colonies which can be obtained in culture using known protocols.

As used herein, “stem” cells are less differentiated forms of progenitor cells. Typically, such cells are often positive for CD34. Some stem cells do not contain this marker, however. CD34+ cells can be assayed using fluorescence activated cell sorting (FACS) and thus their presence can be assessed in a sample using this technique. In general, CD34+ cells are present only in low levels in the blood, but are present in large numbers in bone marrow. While other types of cells such as endothelial cells and mast cells also may exhibit this marker, CD34 is considered an index of stem cell presence.

As used herein, the term “pre-malignant cells” refers to cells that can form malignant hematopoietic or myeloid cells. The malignant hematopoietic or myeloid cells are those which characterize the conditions of myeloma, leukemia, and lymphoma. Particular forms of these diseases include acute myelitic leukemia (AML), acute lymphatic leukemia (ALL), multiple myeloma (MM), chronic myelogenous leukemia (CML), hairy cell leukemia (HCL), acute promyelocytic leukemia (APL), and various lymphomas.

Chemotherapeutic compounds which may be used in the methods whose effectiveness is enhanced by the methods of the invention include carmustine, etoposide, cytarabine, melphalan, cyclophosphamide, busulfan, thiotepa, bleomycin, platinum (cisplatin), cytarabine, cyclophosphamide, buside, cytoxan, daunorubicin, doxorubicin, agent ara-C, cyclosporin; Rituxan®; thalidomide; clofarabine; Velcade®; Antegren®; Ontak®; Revlimid® (thalidomide analog); Prochymal™; Genasense® (oblimersen sodium); Gleevec™; Glivec (imatinib); tamibarotene; nelarabine; gallium nitrate; PT-100; Bexxar®; Zevalin®; pixantrone; Onco-TCS; and agents that are topoisomerase inhibitors, and many others.

A wide variety of chemotherapeutic methods are available in the art. The invention herein employs these standard methods or variations thereof but, in addition, provides for administration of the combinations described above to enhance the effect of such methods. Preferably, the combinations are administered prior to and/or concomitant with subjecting the subject to such methods.

The combination is administered directly to a subject. Each of the essential elements of the combination may be supplied as a single member of the class or may be supplied as a mixture or other combination of the members of the class. Each component of the combination (indeed, each member of the sub-combination representing a single class) can be administered independently, at the same time, by the same route, or at the same time by different routes, or at different times by the same or different routes as any other component in the combination. Thus, for example, if two different CXCR4 antagonists are used, both can be, but need not be, administered at the same time; both can be, but need not be, administered intravenously. Similarly, if two or more VLA-4 antagonist are used, these too may be subject to the variable types of administration just described. The same applies to administration of a member of the CXCR4 antagonist class and a member of the VAL-4 antagonist class. The combination of VLA-4 antagonist(s) and CXCR4 antagonist(s) may also be administered according to such variable protocols, independently or in the same composition.

Compounds Useful in the Invention Method, Formulations and Dosage

VLA-4 antagonists include antibodies, such as humanized monoclonal antibody against α4, natalizumab (Antegren®) and small molecules such as those described in U.S. Pat. No. 5,510,332; WO 06/023396; WO 97/03094; WO 97/02289; WO 96/40781; WO 96/22966; WO 96/20216; WO 96/01644; WO 96/06108; WO 95/15973; WO 96/31206; WO 06/010054; WO 05/087760; WO 01/12186; WO 99/37605; WO 01/51487; WO 03/011288; WO 02/14272; WO 01/32610; and EP 0842943. One useful VLA-4 antagonistic is BIO5192 disclosed in PCT publication WO 01/12186 which has the structure

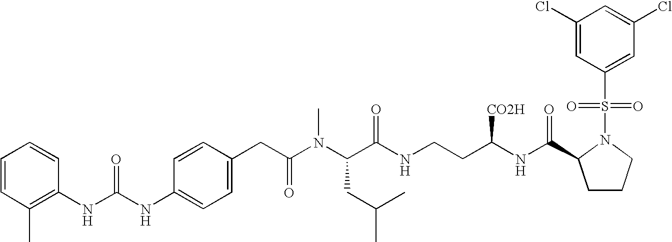

and is sometimes referred to in the present application as AMD15057.

CXCR2 agonists are represented by GROβ and modified forms thereof.

King, A., et al., Blood (2001) 97:1534-1542 have demonstrated that a recombinant N-terminal 4-amino acid truncated form of the human chemokine GROP (also known as SB-251353 or gamocestim) can mobilize progenitor cells after administration of SB-251353 in combination with G-CSF where neutrophils and platelets were mobilized during the studies. Chemokines such as the SB-251353, GROα, GROβ, and GROγ are further discussed in WO 94/29341; WO 97/15594; WO 97/15595; WO 99/26645; WO 02/02132; U.S. Pat. No. 6,080,398; U.S. Pat. No. 6,399,053; and U.S. Pat. No. 6,447,766, all incorporated herein by reference.

SB-251353 is a basic, heparin-binding protein with a molecular mass of approximately 7500 Da, and is a specific CXCR2 receptor agonist (King, A., et al., J. Immunol. (2000) 164:3774-3782, Hepburn, T., et al., Journal of Pharmacology and Experimental Therapeutics, (2001) 298:886-893). Other chemokines, in addition to GROβ, acting via the CXCR2 receptor include GROα, GROγ, GCP-2 (granulocyte chemo-attractant protein 2), IL-8, NAP-2 (neutrophil activating peptide 2), ENA-78 (epithelial-cell derived neutrophil activating protein 78), and MGSA.

The “GROβ protein” or “GROβ chemokine” class includes GROβ itself as well as modified forms of GROβ. These modified forms may be truncated, multimerized, contain amino acid substitutions, deletions or insertions, or may comprise combinations of these.

“Modified forms of GROβ” includes truncated forms thereof, such as those described in U.S. Pat. Nos. 6,447,766; 6,399,053; 6,080,398; PCT publication 99/26645; PCT publication WO 97/15595; PCT publication WO 02/02132; PCT publication WO 97/15594; and PCT publication WO 94/29341. Also included in “modified forms of GROβ” are multimeric forms thereof. Thus “modified forms” include those with truncation of between 2 to about 8 amino acids at the amino terminus of the mature protein, truncation of between about 2 to about 10 amino acids at the carboxy terminus of the mature protein, multimeric forms of the modified and/or truncated proteins, e.g., dimers, trimers, tetramers and other aggregated forms. Truncated forms of GROβ may include SB-251353 which consists of amino acids 5-73 and forms thereof where amino acid 69 is deamidated.

CXCR4 antagonists include AMD3100 and AMD3465. One group of CXCR4 antagonists is exemplified by compounds of the formula:

Z-linker-Z′ (1)

wherein Z is an optionally substituted cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;

or Z is of the formula

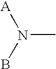

wherein A comprises a monocyclic or bicyclic fused ring system containing at least one N and B is H or an organic moiety of 1-20 atoms;

Z¹ may be embodied in a form as defined by Z above, or alternatively may be of the formula

—N(R)—(CR₂)ₙ—X

wherein each R is independently H or straight, branched or cyclic alkyl (1-6C),

n is 1 or 2, and

X is an aromatic ring, including heteroaromatic rings, or is a mercaptan;

or wherein Z′ can be a nitrogen-containing heterocycle, or can be NR₂ where each R is as defined above; and

“linker” represents a bond, alkylene (1-6C) or may comprise aryl, fused aryl, oxygen atoms contained in an alkylene chain, or may contain keto groups or nitrogen or sulfur atoms.

As described in WO 03/011277, the compounds of formula (1) are used to mobilize and harvest CD34+ cells via apheresis with and without combinations with other mobilizing factors. The harvested cells are used in treatments requiring stem cell transplantations.

In some compounds of formula (1), Z and Z′ are cyclic polyamine moieties having from 9-24C that include 3-5 nitrogen atoms, as described in U.S. Pat. Nos. 5,021,409; 6,001,826 and 5,583,131, incorporated herein by reference. Particularly preferred are 1,5,9,13-tetraazacyclohexadecane; 1,5,8,11,14-pentaazacyclohexadecane; 1,4,8,11-tetraazacylotetradecane; 1,5,9-triazacyclododecane; 1,4,7,10-tetraazacyclododecane; and the like, including such cyclic polyamines which are fused to an additional aromatic or heteroaromatic rings and/or containing a heteroatom other than nitrogen incorporated in the ring. These and embodiments wherein the cyclic polyamine contains a fused additional cyclic system or one or more additional heteroatoms are described in U.S. Pat. No. 5,698,546 incorporated hereinabove by reference. Also preferred are 3,7,11,17-tetraazabicyclo(13.3.1)heptadeca-1(17),13,15-triene; 4,7,10,17-tetraazabicyclo[13.3.1)heptadeca-1(17),13,15-triene; 1,4,7,10-tetraazacyclotetradecane; 1,4,7-triazacyclotetradecane; and 4,7,10-triazabicyclo[13.3.1)heptadeca-1(17),13,15-triene.

When Z′ is other than a cyclic polyamine as defined in Z, its preferred embodiments are set forth in U.S. Pat. Nos. 5,817,807; 6,756,391; 6,506,770; and 6,667,320, also incorporated herein by reference.

Forms where

Z is of the formula

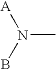

wherein A comprises a monocyclic or bicyclic fused ring system containing at least one N and B is H or an organic moiety of 1-20 atoms are disclosed in U.S. Pat. Nos. 6,734,191; 6,750,348; 6,864,265 and 6,835,731, all incorporated herein by reference.

Preferred forms of the linker moiety include those wherein the linker is a bond, or wherein the linker is an alkylene or includes an aromatic moiety flanked by alkylene, preferably methylene moieties. Preferred linking groups include the methylene bracketed forms of 1,3-phenylene, 2,6-pyridine, 3,5-pyridine, 2,5-thiophene, 4,4′-(2,2′-bipyrimidine); 2,9-(1,10-phenanthroline) and the like. A particularly preferred linker is 1,4-phenylene-bis-(methylene).

Additional compounds that are CXCR4 antagonists are disclosed in U.S. patent application Ser. No. 10/823,494 filed 12 Apr. 2004, and U.S. Patent Publication Nos. U.S-2005-0059702-A1 and US-2005-0277670-A1, incorporated herein by reference.

Embodiments of the compound of the formula (1) include 2,2′-bicyclam; 6,6′-bicyclam; the embodiments set forth in U.S. Pat. Nos. 5,021,409, and 6,001,826, and in particular 1,1′-[1,4-phenylene-bis(methylene)]-bis-1,4,8,11-tetraazacyclotetradecane, set forth in U.S. Pat. No. 5,583,131, and designated herein AMD3100. Also preferred are N′-(1H-benzimidazol-2-ylmethyl)-N′-(5,6,7,8-tetrahydroquinoline-8-yl)-butane-1,4-diamine as described in U.S. Patent Publication No. US-2003-9229341-A1. A list of specific embodiments of Formula (1) is set forth after the Examples section herein as Appendix A.

Methods to synthesize the compounds of Formula (1) useful in the method of the invention are set forth in the U.S. patents and applications above as well as U.S. Pat. No. 6,489,472 and U.S. Patent Publication No. US-2005-0209277-A1, incorporated herein by reference. Additional CXCR4 inhibitors are set forth in Appendix B.

Other CXCR4 inhibitors that may be used to practice the methods of the invention include but are not limited to CTCE-0214; CTCE-9908; CP-1221 (linear peptides, cyclic peptides, natural amino-acids, unnatural amino acids, and peptidomimetic compounds); T140 and analogs; 4F-benzoyl-TN24003; KRH-1120; KRH-1636; KRH-2731; polyphemusin analogue; ALX40-4C; or those described in WO 01/85196; WO 99/50461; WO 01/94420; WO 03/090512, each of which is incorporated by reference herein.

As stated above, the compounds of formula (1) are employed in combination with other VLA-4 antagonists such as those described in U.S. Pat. No. 5,510,332; WO 06/023396; WO 97/03094; WO 97/02289; WO 96/40781; WO 96/22966; WO 96/20216; WO 96/01644; WO 96/06108; WO 95/15973; WO 96/31206; WO 06/010054; WO 05/087760; WO 01/12186; WO 99/37605; WO 01/51487; WO 03/011288; WO 02/14272; WO 01/32610; EP 0842943; and natalizumab (or Antegren®).

The compounds useful in the invention may be prepared in the form of prodrugs, i.e., protected forms which release the compounds of the invention after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2 ed., London (1988).

Compounds useful in the invention which are amines, may be administered or prepared in the forms of their acid addition salts or metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Typically, at physiological pH, the compounds of the invention will be in the forms of the acid addition salts.

Compounds useful in the invention that are carboxylic acids or otherwise acidic may be administered or prepared in forms of salts formed from inorganic or organic bases that are physiologically compatible. Thus, these compounds may be prepared in the forms of their sodium, potassium, calcium, or magnesium salts as appropriate or may be salts with organic bases such as caffeine or ethylamine. These compounds also may be in the form of metal complexes.

When prepared as purified forms, the compounds may also be crystallized as the hydrates or other solvates. Those forms of the compounds used in the invention that contain chiral centers may be optically pure or may contain a mixture of stereoisomers, including racemic mixtures or mixtures of varying optical purity.

The combinations of the invention may also include additional active ingredients that are therapeutically or nutritionally useful such as antibiotics, vitamins, herbal extracts, anti-inflammatories, glucose, antipyretics, analgesics, cyclophosphamide, recombinant G-CSF (Neupogen®, Granocyte®/Neutrogin®, and Stemgen®), and covalent conjugate of recombinant G-CSF (Neulasta®) granulocyte-macrophage colony stimulating factor (GM-CSF) (such as Leukine®, and Leucomax®), ETRX-101, TLK 199/TILENTRA™, Interleukin-1 (IL-1), Interleukin-3 (IL-3), Interleukin-8 (IL-8), PIXY-321 (GM-CSF/IL-3 fusion protein), macrophage inflammatory protein, stem cell factor, thrombopoietin, and the like.

Formulations for administration to animal subject use commonly understood formulation techniques well known in the art. Formulations which are suitable for particular modes of administration and for compounds of the type represented by those of formula (1) may be found in Remington's Pharmaceutical Sciences, latest edition, Mack Publishing Company, Easton, Pa.; similarly, methods for administering polypeptides such as those represented by VLA-4 antagonist thereof are found in this source.

Preferably, the compounds are administered by injection, such as by intravenous injection, but also by subcutaneous or intraperitoneal injection, and the like. Additional parenteral routes of administration include intramuscular and intraarticular injection. For intravenous or parenteral administration, the compounds are formulated in suitable liquid form with excipients as required. The compositions may contain liposomes or other suitable carriers. For injection intravenously, the solution is made isotonic using standard preparations such as Hank's solution.

Besides injection, other routes of administration may also be used. The compounds may be formulated into tablets, capsules, syrups, powders, or other suitable forms for administration orally. By using suitable excipients, these compounds may also be administered through the mucosa using suppositories or intranasal sprays. Transdermal administration can also be effected by using suitable penetrants and controlling the rate of release.

The formulation and route of administration chosen will be tailored to the individual subject, the nature of the condition to be treated in the subject, and generally, the judgment of the attending practitioner.

Suitable dosage ranges for the CXCR4 inhibitor, CXCR2 agonist and VLA-4 inhibitor vary according to these considerations, but in general, the compounds when administered alone are administered in the range of about 0.1 μg/kg-5 mg/kg of body weight; preferably the range is about 1 μg/kg-300 μg/kg of body weight; more preferably about 10 μg/kg-100 μg/kg of body weight. In some embodiments, the dose is about 240 μg per 1 kg, especially for AMD3100. For a typical 70-kg human subject, thus, the dosage range is from about 0.7 μg-350 mg. The combination of at least one CXCR4 inhibitor and the at least one VLA-4 antagonist (or at least one CXCR2 agonist) composition may be administered together in a single formulation, simultaneously in separate formulations by the same or different routes, or at staggered times, again by the same or different routes. Optimization of the protocols for administration to a particular subject is well within ordinary skill. The combination may be administered as a single bolus dose, a dose over time, as in i.v. or transdermal administration, or in multiple dosages. One protocol includes once daily for 2-4 days

Subjects that will respond favorably to the method of the invention include medical and veterinary subjects generally, including human patients. Among other subjects for whom the methods of the invention is useful are cats, dogs, large animals, avians such as chickens, and the like. In general, any subject who would benefit from an elevation of progenitor cells and/or stem cells, or whose progenitor cells and/or stem cells are desirable for stem cell transplantation are appropriate for the invention method. Other suitable subjects include subjects with multiple myeloma or hematopoietic malignancies.

Applications of Combination Treatment

The combination treatment of the invention is useful in a number of contexts. In one embodiment, the combination is able to mobilize stem and/or progenitor cells from bone marrow into the circulation where the mobilized cells may either be harvested or may remain in the subject so as to effect tissue repair, in particular repair of myocardial tissue. The administration of the combination may also result in mobilizing leukemic or other white blood cells into the circulation to make them more accessible to radiation or chemotherapy. Methods to effect this mobilization and treatment are described in detail in U.S. Ser. No. 60/709,978 filed 19 Aug. 2005 and U.S. Ser. No. 60/734,736 filed 8 Nov. 2005. The contents of these applications are incorporated herein by reference.

If the cells are harvested, they may be returned to the donor subject (autologous transplant) or may be donated to another subject that is sufficiently compatible to prevent rejection (allogeneic transplant). A common application of autologous transplantation is in combination with radiation or chemotherapy in subjects bearing tumors since the radiotherapeutic or chemotherapeutic methods deplete wanted normal cells. In this application, the subjects cells may be harvested prior to or during the therapeutic treatments, fractionated if necessary, cultured and optionally expanded, and then returned to the subject to restore the damaged immune system depleted by the therapy. Allogeneic recipients may receive the cells for the same purpose, or may have a condition that may be benefited by enhancing their hematopoietic systems.

In a typical protocol, the mobilized cells are collected from the donor by, for example, apheresis and then stored/cultured/expanded/fractionated as desired. A particular advantage of the present invention is that the time required for harvest of the progenitor and/or stem cells is demonstrably shortened as compared to alternative methods of mobilization.

In lieu of harvesting the cells from the donor, the mobilization effected by administering the combination may be used internally for tissue repair. Thus, the circulating progenitor cells are allowed to home to a tissue in need of repair, such as a myocardial tissue to restore function.

In addition to mobilizing progenitor and/or stem cells for harvest or for internal tissue repair, the combinations described herein may be used to treat multiple myeloma (MM). While not wishing to be bound by any theory, it appears that the combination effects mobilization of tumor cells from the bone marrow or, alternatively, prevents engraftment of the tumor cells in the bone. The protocols for administration of MM treatment are similar to those with respect to progenitor/stem cell mobilization.

Having now generally described the invention, the same will be more readily understood through reference to the following examples, which are provided by way of illustration, and do not limit the invention.

### EXAMPLE 1

Compounds are administered i.v. at doses of 1 mg/kg (15057) and 2 mg/kg (3100) either as single agents or as combination. AMD15057, a VLA-4 inhibitor, is formulated at a concentration of 0.2 mg/ml, and AMD3100 is formulated at a concentration of 0.4 mg/ml. The vehicle is 36:45:10 PG/water/ethanol at pH 6.6. Blood samples are taken at appropriate time intervals and measurements made including white blood cell counts and levels of progenitor cells by colony forming assays.

### EXAMPLE 2

### Mobilization of Leukemic Cells

A mouse model of human acute promyelocytic leukemia (APL) was employed wherein PML-RARa transgene was knocked into a single allele of the murine cathepsin G locus. To more efficiently track the leukemic cells, banked APL tumors were transduced with a dual function reporter gene that encodes a fusion protein comprised of click beetle red (CBR) luciferase which is a bioluminescence imaging (BLI) optical reporter gene, and EGFP for ex vivo cell sorting (CBR/EGFP). Large numbers of CBR/EGFP+ APL cells were generated by isolating EGFP+ cells using a MoFlo™ cell sorter, and passaging them in secondary syngeneic recipients. The secondary recipients developed a rapidly fatal acute leukemia after intravenous or intraperitoneal injection of these cells, i.e., cells that displayed an APL phenotype (CD34/GR1 co-expression) and exhibited luciferase activity. Upon i.v. injection into syngeneic recipients, the CBR/EGFP+ APL cells rapidly migrated to the bone marrow (BM) microenvironment, as evidenced by the significantly increased BLI signal in the femurs, spine, ribs, and skull of recipients at 4 days after injection. Over the next 2-3 days the CBR/EGFP+ cells migrated to the spleen followed rapidly by widespread dissemination and death due to leukostasis by 14-16 days. To our knowledge, this represents the only mouse leukemia model in which leukemia cells home preferentially to the BM microenvironment in a manner that is similar to what is seen in human AML.

The effect of the combination of AMD3100 and AMD15057 on the “mobilization” of APL cells into the peripheral blood and on their sensitivity to chemotherapeutic agents that are known to affect the proliferation of these cells is studied. AMD3100 (5 mg/kg) and AMD15057 are injected at the time of APL infusion and 11 days after APL injection. The impact on the engraftment (short term or long term) of either normal BM stem cells or the leukemic cells is then determined. Mobilization of the leukemic cells is observed. The combination is also administered concomitant with cytarabine (AraC) (200 mg/kg) and the overall survival of mice is prolonged, compared with mice treated only with AraC.

Similarly, following the procedure in the previous paragraph, the effect of the combination of AMD3100 and SB-251353 is tested on the mobilization of APL cells into the peripheral blood and their sensitivity to chemotherapeutic agents.

### APPENDIX A

Exemplary Suitable CXCR4 Inhibitors Include
N-[1,4,8,11-tetraazacyclotetradecanyl-1,4-phenylenebis(methylene)]-2-(amino-methyl)pyridine;
N-[1,4,8,11-tetraazacyclotetradecanyl-1,4-phenylenebis(methylene)]-N-methyl-2-(aminomethyl)pyridine;
N-[1,4,8,11-tetraazacyclotetradecanyl-1,4-phenylenebis(methylene)]-4-(amino-methyl)pyridine;
N-[1,4,8,11-tetraazacyclotetradecanyl-1,4-phenylenebis(methylene)]-3-(amino-methyl)pyridine;
N-[1,4,8,11-tetraazacyclotetradecanyl-1,4-phenylenebis(methylene)]-(2-amino-methyl-5-methyl)pyrazine; and
N-[1,4,8,11-tetraazacyclotetradecanyl-1,4-phenylenebis(methylene)]-2-(amino-ethyl)pyridine; described in U.S. Pat. No. 6,667,320 referenced above.
N-[1,4,8,11-tetraazacyclotetradecanyl-1,4-phenylenebis(methylene)]-2-aminomethyl)pyridine;
7,7′-[1,4-phenylenebis(methylene)]bis-4,7,10,17-tetraazabicyclo-[13.3.1]heptadeca-1(17),13,15-triene;

7,7′-[1,4-phenylenebis(methylene)]bis-3,7,11,17-tetraazabicyclo[13.3.1]heptadeca-1(17),13,15-triene;
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane;
1,1′-[1,4-phenylene-bis-(methylene)]-bis-1,4,7,10-tetraazacyclotetradecane;
1,1′-[1,3-phenylene-bis-(methylene)]-bis-1,4,7,10-tetraazacyclotetradecane;
11,11′-(1,2-propanediyl)bis-1,4,8,11-tetraazacyclotetradecane;
N-[4-(1,4,7-triazacyclotetra-decane)-1,4-phenylenebis(methylene)]-2-(aminomethyl)pyridine;
N-[7-(4,7,10-triazabicyclo[13.3.1]heptadeca-1(17),13,15-triene)-1,4-phenylenebis(methylene)]-2-(aminomethyl)pyridine;
N-[7-(4,7,10,17-tetraazabicyclo[13.3.1]heptadeca-1(17),13,15-triene)-1,4-phenylenebis(methylene)]-2-(aminomethyl)pyridine;
N-[4-[4,7,10,17-tetraazabicyclo[13.3.1]heptadeca-1(17),13,15-triene]-1,4-phenylenebis(methylene)]-2-(aminomethyl)pyridine;
3,3′-(bis-1,5,9,13-tetraazacyclohexadecane);
3,3′-(bis-1,5,8,11,14-pentaazacyclohexadecane), methylene (or polymethylene) di-1-N-1,4,8,11-tetraazacyclotetradecane;
3,3′-bis-1,5,9,13,-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis(methylene)]-1,4,8,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(11,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenylenebis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene;
17,17′-[1,4-phenylene-bis(methylene)]bis-3,6,14,17,23,24-hexaazatricyclo[17.3.1.18,12]tetracosa-1(23),8,10,12(24),19,21-hexaene;
N-[1,4,8,11-Tetraazacyclotetradecanyl-1,4-phenylenebis(methylene)]-2-(amino-methyl)thiophene;
N-[1,4,8,11-Tetraazacyclotetradecanyl-1,4-phenylenebis(methylene)]-2-(amino-ethyl)mercaptan;
N-[1,4,8,11-Tetraazacyclotetradecanyl-1,4-phenylenebis(methylene)]-2-amino-benzylamine;
N-[1,4,8,11-Tetraazacyclotetradecanyl-1,4-phenylenebis(methylene)]-4-amino-benzylamine;
N-[1,4,8,11-Tetraazacyclotetradecanyl-1,4-phenylenebis(methylene)]-4-(amino-ethyl)imidazole;
N-[1,4,8,11-Tetraazacyclotetradecanyl-1,4-phenylenebis(methylene)]-benzylamine;
N-[1,4,8,11-Tetraazacyclotetradecanyl-1,4-phenylenebis(methylene)]-purine;
N-[1,4,8,11-Tetraazacyclotetradecanyl-1,4-phenylenebis(methylene)]-4-phenylpiperazine;
N-[4-(1,4,7-Triazacyclotetra-decanyl)-1,4-phenylenebis(methylene)]-2-(aminomethyl)pyridine;
N-[7-(4,7,10,17-Tetraazabicyclo[13.3.1]heptadeca-1(17),13,15-trienyl)-1,4-phenylenebis(methylene)]-2-(aminomethyl)pyridine;
N-[7-(4,7,10-Triazabicyclo[13.3.1]heptadeca-1(17),13,15-trienyl)-1,4-phenylenebis(methylene)]-2-(aminomethyl)pyridine;
N-[4-[4,7,10-Triazabicyclo[13.3.1]heptadeca-1(17),13,15-trienyl]-1,4-phenylenebis(methylene)]-2-(aminomethyl)pyridine;
N-[1-(1,4,7-Triazacyclotetra-decanyl)-1,4-phenylenebis(methylene)]-2-(aminomethyl)pyridine;
N-[4-[4,7,10,17-Tetraazabicyclo[13.3.1]heptadeca-1(17),13,15-trienyl]-1,4-phenylenebis(methylene)]-2-(aminomethyl)pyridine;
N-[3-(3,6,17-Triazabicyclo[13.3.1]heptadeca-1(17),13,15-trienyl)-1,4-phenylenebis(methylene)]-2-(aminomethyl)pyridine;
N-[3-(3,6,17-Triazabicyclo[13.3.1]heptadeca-1(17),13,15-trienyl)-1,3-phenylenebis(methylene)]-2-(aminomethyl)pyridine;
N-[4-(4,7,17-Triazabicyclo[13.3.1]heptadeca-1(17),13,15-trienyl)-1,4-phenylenebis(methylene)]-2-(aminomethyl)pyridine;
N-[7-(4,7,17-Triazabicyclo[13.3.1]heptadeca-1(17),13,15-trienyl)-1,4-phenylenebis(methylene)]-2-(aminomethyl)pyridine;
N-[6-(3,6,9-Triazabicyclo[11.3.1]pentadeca-[(15),11,13-trienyl)-1,3-phenylenebis(methylene)]-2-(aminomethyl)pyridine;
N-[7-(4,10,17-Triazabicyclo[13.3.1]heptadeca-1(17),13,15-trienyl)-1,4-phenylenebis(methylene)]-2-(aminomethyl)pyridine;
N-[4-(1,7-Diazacyclotetradecanyl)-1,4-phenylenebis(methylene)]-2-(aminomethyl)pyridine;
N-[7-(4,10-Diazabicyclo[13.3.1]heptadeca-1(17),13,15-trienyl)-1,4-phenylenebis(methylene)]-2-(aminomethyl)pyridine;
N-[4-(11-Fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-one)-yl))-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,7-diazacyclotetradecanyl)-1,4-phenylenebis(methylene)]-2-(aminomethyl)pyridine;
N-[4-(11-thia-1,7-diazacyclotetradecanyl)-1,4-phenylenebis(methylene)]-2-(aminomethyl)pyridine;
N-[4-(11-sulfoxo-1,7-diazacyclotetradecanyl)-1,4-phenylenebis(methylene)]-2-(aminomethyl)pyridine;
N-[4-(11-sulfono-1,7-diazacyclotetradecanyl)-1,4-phenylenebis(methylene)]-2-(aminomethyl)pyridine;
N-[4-(1,4,7-triazacyclotetradecan-3-one)-yl))-1,4-phenylenebis(methylene)]-2-(aminomethyl)pyridine;
N-(2-pyridinylmethyl)-N′-(6,7,8,9-tetrahydro-5H-cyclohepta[b]pyridin-9-yl)-1,4-benzenedimethanamine;
N-(2-pyridinylmethyl)-N′-(5,6,7,8-tetrahydro-8-quinolinyl)-1,4-benzenedimethanamine;
N-(2-pyridinylmethyl)-N′-(6,7-dihydro-5H-cyclopenta[b]pyridin-7-yl)-1,4-benzenedimethanamine;
N-(2-pyridinylmethyl)-N′-(1,2,3,4-tetrahydro-1-naphthalenyl)-1,4-benzenedimethanamine;
N-(2-pyridinylmethyl)-N′-(1-naphthalenyl)-1,4-benzenedimethanamine;
N-(2-pyridinylmethyl)-N′-(8-quinolinyl)-1,4-benzenedimethanamine;
N-(2-pyridinylmethyl)-N′-[2-[(2-pyridinylmethyl)amino]ethyl]-N′-(1-methyl-1,2,3,4-tetrahydro-8-quinolinyl)-1,4-benzenedimethanamine;
N-(2-pyridinylmethyl)-N′-[2-[(1H-imidazol-2-ylmethyl)amino]ethyl]-N′-(1-methy 1-1,2,3,4-tetrahydro-8-quinolinyl)-1,4-benzenedimethanamine;
N-(2-pyridinylmethyl)-N′-(1,2,3,4-tetrahydro-8-quinolinyl)-1,4-benzenedimethanamine;
N-(2-pyridinylmethyl)-N′-[2-[(1H-imidazol-2-ylmethyl)amino]ethyl]-N′-(1,2,3,4-tetrahydro-1-naphthalenyl)-1,4-benzenedimethanamine;
N-(2-pyridinylmethyl)-N′-(2-phenyl-5,6,7,8-tetrahydro-8-quinolinyl)-1,4-benzenedimethanamine;
N,N′-bis(2-pyridinylmethyl)-N′-(2-phenyl-5,6,7,8-tetrahydro-8-quinolinyl)-1,4-benzenedimethanamine;
N-(2-pyridinylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-8-quinolinyl)-1,4-benzenedimethanamine;
N-(2-pyridinylmethyl)-N′-[(2-amino-3-phenyl)propyl]-N′-(5,6,7,8-tetrahydro-8-quinolinyl)-1,4-benzenedimethanamine;
N-(2-pyridinylmethyl)-N′-(1H-imidazol-4-ylmethyl)-N′-(5,6,7,8-tetrahydro-8-quinolinyl)-1,4-benzenedimethanamine;
N-(2-pyridinylmethyl)-N′-(2-quinolinylmethyl)-N′-(5,6,7,8-tetrahydro-8-quinolinyl)-1,4-benzenedimethanamine;
N-(2-pyridinylmethyl)-N′-(2-(2-naphthoyl)aminoethyl)-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[3-((2-naphthalenylmethyl)amino)propyl]-N′-(5,6,7,8-tetrahydro-8-quinolinyl)-1,4-benzenedimethanamine;
N-(2-pyridinylmethyl)-N′-[2-(S)-pyrollidinylmethyl]-N′-(5,6,7,8-tetrahydro-8-quinolinyl)-1,4-benzenedimethanamine;
N-(2-pyridinylmethyl)-N′-[2-(R)-pyrollidinylmethyl]-N′-(5,6,7,8-tetrahydro-8-quinolinyl)-1,4-benzenedimethanamine;
N-(2-pyridinylmethyl)-N′-[3-pyrazolylmethyl]-N′-(5,6,7,8-tetrahydro-8-quinolinyl)-1,4-benzenedimethanamine;
N-(2-pyridinylmethyl)-N′-[2-pyrrolylmethyl]-N′-(5,6,7,8-tetrahydro-8-quinolinyl)-1,4-benzenedimethanamine;
N-(2-pyridinylmethyl)-N′-[2-thiopheneylmethyl]-N′-(5,6,7,8-tetrahydro-8-quinolinyl)-1,4-benzenedimethanamine

N-(2-pyridinylmethyl)-N′-[2-thiazolylmethyl]-N′-(5,6,7,8-tetrahydro-8-quinolinyl)-1,4-benzenedimethanamine;
N-(2-pyridinylmethyl)-N′-[2-furanylmethyl]-N′-(5,6,7,8-tetrahydro-8-quinolinyl)-1,4-benzenedimethanamine;
N-(2-pyridinylmethyl)-N′-[2-[(phenylmethyl)amino]ethyl]-N′-(5,6,7,8-tetrahydro-8-quinolinyl)-1,4-benzenedimethanamine;
N-(2-pyridinylmethyl)-N′-(2-aminoethyl)-N′-(5,6,7,8-tetrahydro-8-quinolinyl)-1,4-benzenedimethanamine;
N-(2-pyridinylmethyl)-N′-3-pyrrolidinyl-N′-(5,6,7,8-tetrahydro-8-quinolinyl)-1,4-benzenedimethanamine

N-(2-pyridinylmethyl)-N′-4-piperidinyl-N′-(5,6,7,8-tetrahydro-8-quinolinyl)-1,4-benzenedimethanamine;
N-(2-pyridinylmethyl)-N′-[2-[(phenyl)amino]ethyl]-N′-(5,6,7,8-tetrahydro-8-quinolinyl)-1,4-benzenedimethanamine;
N-(2-pyridinylmethyl)-N′-(7-methoxy-1,2,3,4-tetrahydro-2-naphthalenyl)-1,4-benzenedimethanamine;
N-(2-pyridinylmethyl)-N′-(6-methoxy-1,2,3,4-tetrahydro-2-naphthalenyl)-1,4-benzenedimethanamine;
N-(2-pyridinylmethyl)-N′-(1-methyl-1,2,3,4-tetrahydro-2-naphthalenyl)-1,4-benzenedimethanamine;
N-(2-pyridinylmethyl)-N′-(7-methoxy-3,4-dihydronaphthalenyl)-1-(aminomethyl)-4-benzamide;
N-(2-pyridinylmethyl)-N′-(6-methoxy-3,4-dihydronaphthalenyl)-1-(aminomethyl)-4-benzamide;
N-(2-pyridinylmethyl)-N′-(1H-imidazol-2-ylmethyl)-N′-(7-methoxy-1,2,3,4-tetrahydro-2-naphthalenyl)-1,4-benzenedimethanamine;
N-(2-pyridinylmethyl)-N′-(8-hydroxy-1,2,3,4-tetrahydro-2-naphthalenyl)-1,4-benzenedimethanamine;
N-(2-pyridinylmethyl)-N′-(1H-imidazol-2-ylmethyl)-N′-(8-hydroxy-1,2,3,4-tetrahydro-2-naphthalenyl)-1,4-benzenedimethanamine;
N-(2-pyridinylmethyl)-N′-(8-Fluoro-1,2,3,4-tetrahydro-2-naphthalenyl)-1,4-benzenedimethanamine;
N-(2-pyridinylmethyl)-N′-(1H-imidazol-2-ylmethyl)-N′-(8-Fluoro-1,2,3,4-tetrahydro-2-naphthalenyl)-1,4-benzenedimethanamine;
N-(2-pyridinylmethyl)-N′-(5,6,7,8-tetrahydro-7-quinolinyl)-1,4-benzenedimethanamine;
N-(2-pyridinylmethyl)-N′-(1H-imidazol-2-ylmethyl)-N′-(5,6,7,8-tetrahydro-7-quinolinyl)-1,4-benzenedimethanamine;
N-(2-pyridinylmethyl)-N′-[2-[(2-naphthalenylmethyl)amino]ethyl]-N′-(5,6,7,8-tetrahydro-8-quinolinyl)-1,4-benzenedimethanamine;
N-(2-pyridinylmethyl)-N′-[2-(isobutylamino)ethyl]-N′-(5,6,7,8-tetrahydro-8-quinolinyl)-1,4-benzenedimethanamine;
N-(2-pyridinylmethyl)-N′-[2-[(2-pyridinylmethyl)amino]ethyl]-N′-(5,6,7,8-tetrahydro-8-quinolinyl)-1,4-benzenedimethanamine;
N-(2-pyridinylmethyl)-N′-[2-[(2-furanylmethyl)amino]ethyl]-N′-(5,6,7,8-tetrahydro-8-quinolinyl)-1,4-benzenedimethanamine;
N-(2-pyridinylmethyl)-N′-(2-guanidinoethyl)-N′-(5,6,7,8-tetrahydro-8-quinolinyl)-1,4-benzenedimethanamine;
N-(2-pyridinylmethyl)-N′-[2-[bis-[(2-methoxy)phenylmethyl]amino]ethyl]-N′-(5,6,7,8-tetrahydro-8-quinolinyl)-1,4-benzenedimethanamine;
N-(2-pyridinylmethyl)-N′-[2-[(1H-imidazol-4-ylmethyl)amino]ethyl]-N′-(5,6,7,8-tetrahydro-8-quinolinyl)-1,4-benzenedimethanamine;
N-(2-pyridinylmethyl)-N′-[2-[(1H-imidazol-2-ylmethyl)amino]ethyl]-N′-(5,6,7,8-tetrahydro-8-quinolinyl)-1,4-benzenedimethanamine;
N-(2-pyridinylmethyl)-N′-[2-(phenylureido)ethyl]-N′-(5,6,7,8-tetrahydro-8-quinolinyl)-1,4-benzenedimethanamine;
N-(2-pyridinylmethyl)-N′-[[N″-(n-butyl)carboxamido]methyl]-N′-(5,6,7,8-tetrahydro-8-quinolinyl)-1,4-benzenedimethanamine;
N-(2-pyridinylmethyl)-N′-(carboxamidomethyl)-N′-(5,6,7,8-tetrahydro-8-quinolinyl)-1,4-benzenedimethanamine;
N-(2-pyridinylmethyl)-N′-[(N″-phenyl)carboxamidomethyl]-N′-(5,6,7,8-tetrahydro-8-quinolinyl)-1,4-benzenedimethanamine;
N-(2-pyridinylmethyl)-N′-(carboxymethyl)-N′-(5,6,7,8-tetrahydro-8-quinolinyl)-1,4-benzenedimethanamine;
N-(2-pyridinylmethyl)-N′-(phenylmethyl)-N′-(5,6,7,8-tetrahydro-8-quinolinyl)-1,4-benzenedimethanamine;
N-(2-pyridinylmethyl)-N′-(1H-benzimidazol-2-ylmethyl)-N′-(5,6,7,8-tetrahydro-8-quinolinyl)-1,4-benzenedimethanamine;
N-(2-pyridinylmethyl)-N′-(5,6-dimethyl-1H-benzimidazol-2-ylmethyl)-N′-(5,6,7,8-tetrahydro-8-quinolinyl)-1,4-benzenedimethanamine (hydrobromide salt);
N-(2-pyridinylmethyl)-N′-(5-nitro-1H-benzimidazol-2-ylmethyl)-N′-(5,6,7,8-tetrahydro-8-quinolinyl)-1,4-benzenedimethanamine;
N-(2-pyridinylmethyl)-N′-[(1H)-5-azabenzimidazol-2-ylmethyl]-N′-(5,6,7,8-tetrahydro-8-quinolinyl)-1,4-benzenedimethanamine;
N-(2-pyridinylmethyl)-N-(4-phenyl-1H-imidazol-2-ylmethyl)-N′-(5,6,7,8-tetrahydro-8-quinolinyl)-1,4-benzenedimethanamine;
N-(2-pyridinylmethyl)-N′-[2-(2-pyridinyl)ethyl]-N′-(5,6,7,8-tetrahydro-8-quinolinyl)-1,4-benzenedimethanamine;
N-(2-pyridinylmethyl)-N′-(2-benzoxazolyl)-N′-(5,6,7,8-tetrahydro-8-quinolinyl)-1,4-benzenedimethanamine;
N-(2-pyridinylmethyl)-N′-(trans-2-aminocyclohexyl)-N′-(5,6,7,8-tetrahydro-8-quinolinyl)-1,4-benzenedimethanamine;
N-(2-pyridinylmethyl)-N′-(2-phenylethyl)-N′-(5,6,7,8-tetrahydro-8-quinolinyl)-1,4-benzenedimethanamine;
N-(2-pyridinylmethyl)-N′-(3-phenylpropyl)-N′-(5,6,7,8-tetrahydro-8-quinolinyl)-1,4-benzenedimethanamine;
N-(2-pyridinylmethyl)-N′-(trans-2-aminocyclopentyl)-N′-(5,6,7,8-tetrahydro-8-quinolinyl)-1,4-benzenedimethanamine;
N-[[4-[[(2-pyridinylmethyl)amino]methyl]phenyl]methyl]-N-(5,6,7,8-tetrahydro-8-quinolinyl)-glycinamide;
N-[[4-[[(2-pyridinylmethyl)amino]methyl]phenyl]methyl]-N-(5,6,7,8-tetrahydro-8-quinolinyl)-(L)-alaninamide;
N-[[4-[[(2-pyridinylmethyl)amino]methyl]phenyl]methyl]-N-(5,6,7,8-tetrahydro-8-quinolinyl)-(L)-aspartamide;
N-[[4-[[(2-pyridinylmethyl)amino]methyl]phenyl]methyl]-N-(5,6,7,8-tetrahydro-8-quinolinyl)-pyrazinamide;
N-[[4-[[(2-pyridinylmethyl)amino]methyl]phenyl]methyl]-N-(5,6,7,8-tetrahydro-8-quinolinyl)-(L)-prolinamide;
N-[[4-[[(2-pyridinylmethyl)amino]methyl]phenyl]methyl]-N-(5,6,7,8-tetrahydro-8-quinolinyl)-(L)-lysinamide;
N-[[4-[[(2-pyridinylmethyl)amino]methyl]phenyl]methyl]-N-(5,6,7,8-tetrahydro-8-quinolinyl)-benzamide;
N-[[4-[[(2-pyridinylmethyl)amino]methyl]phenyl]methyl]-N-(5,6,7,8-tetrahydro-8-quinolinyl)-picolinamide;
N′-Benzyl-N-[[4-[[(2-pyridinylmethyl)amino]methyl]phenyl]methyl]-N-(5,6,7,8-tetrahydro-8-quinolinyl)-urea;
N′-phenyl-N-[[4-[[(2-pyridinylmethyl)amino]methyl]phenyl]methyl]-N-(5,6,7,8-tetrahydro-8-quinolinyl)-urea;
N-(6,7,8,9-tetrahydro-5H-cyclohepta[bacteriapyridin-9-yl)-4-[[(2-pyridinylmethyl)amino]methyl]benzamide;
N-(5,6,7,8-tetrahydro-8-quinolinyl)-4-[[(2-pyridinylmethyl)amino]methyl]benzamide;
N,N′-bis(2-pyridinylmethyl)-N′-(5,6,7,8-tetrahydro-8-quinolinyl)-1,4-benzenedimethanamine;
N,N′-bis(2-pyridinylmethyl)-N′-(6,7,8,9-tetrahydro-5H-cyclohepta[bacteriapyridin-9-yl)-1,4-benzenedimethanamine;
N,N′-bis(2-pyridinylmethyl)-N′-(6,7-dihydro-5H-cyclopenta[bacteriapyridin-7-yl)-1,4-benzenedimethanamine;
N,N′-bis(2-pyridinylmethyl)-N′-(1,2,3,4-tetrahydro-1-naphthalenyl)-1,4-benzenedimethanamine;
N,N′-bis(2-pyridinylmethyl)-N′-[(5,6,7,8-tetrahydro-8-quinolinyl)methyl]-1,4-benzenedimethanamine;
N,N′-bis(2-pyridinylmethyl)-N′[(6,7-dihydro-5H-cyclopenta[bacteriapyridin-7-yl)methyl]-1,4-benzenedimethanamine;
N-(2-pyridinylmethyl)-N-(2-methoxyethyl)-N′-(5,6,7,8-tetrahydro-8-quinolinyl)-1,4-benzenedimethanamine;
N-(2-pyridinylmethyl)-N-[2-(4-methoxyphenyl)ethyl]-N′-(5,6,7,8-tetrahydro-8-quinolinyl)-1,4-benzenedimethanamine;
N,N′-bis(2-pyridinylmethyl)-1,4-(5,6,7,8-tetrahydro-8-quinolinyl)benzenedimethanamine;
N-[(2,3-dimethoxyphenyl)methyl]-N′-(2-pyridinylmethyl)-N-(5,6,7,8-tetrahydro-8-quinolinyl)-1,4-benzenedimethanamine;
N,N′-bis(2-pyridinylmethyl)-N-[1-(N″-phenyl-N″-methylureido)-4-piperidinyl]-1,3-benzenedimethanamine;
N,N′-bis(2-pyridinylmethyl)-N-[N″-p-toluenesulfonylphenylalanyl)-4-piperidinyl]-1,3-benzenedimethanamine;
N,N′-bis(2-pyridinylmethyl)-N-[1-[3-(2-chlorophenyl)-5-methyl-isoxazol-4-oyl]-4-piperidinyl]-1,3-benzenedimethanamine;
N-[(2-hydroxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(5,6,7,8-tetrahydro-8-quinolinyl)-1,4-benzenedimethanamine;
N-[(4-acetamidophenyl)methyl]-N′-(2-pyridinylmethyl)-N-(5,6,7,8-tetrahydro-8-quinolinyl)-1,4-benzenedimethanamine;
N-[(4-phenoxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(1-methyl-2-carboxamido)ethyl]-N,N′-bis(2-pyridinylmethyl)-1,3-benzenedimethanamine;
N-[(4-benzyloxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(thiophene-2-yl)methyl]-N′-(2-pyridinylmethyl)-N-(6,7,8,9-tetrahydro-5H-cyclohepta[bacteriapyridin-9-yl)-1,4-benzenedimethanamine;
N-[1-(benzyl)-3-pyrrolidinyl]-N,N′-bis(2-pyridinylmethyl)-1,3-benzenedimethanamine;
N-[[1-methyl-3-(pyrazol-3-yl)]propyl]-N,N′-bis(2-pyridinylmethyl)-1,3-benzenedimethanamine;
N-[1-(phenyl)ethyl]-N,N′-bis(2-pyridinylmethyl)-1,3-benzenedimethanamine;
N-[(3,4-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[1-benzyl-3-carboxymethyl-4-piperidinyl]-N,N′-bis(2-pyridinylmethyl)-1,3-benzenedimethanamine;
N-[(3,4-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(6,7,8,9-tetrahydro-5H-cyclohepta[b]pyridin-9-yl)-1,4-benzenedimethanamine;

N-[[1-methyl-2-(2-tolyl)carboxamido]ethyl]-N,N′-bis(2-pyridinylmethyl)-1,3-benzenedimethanamine;
N-[(1,5-dimethyl-2-phenyl-3-pyrazolinone-4-yl)methyl]-N′-(2-pyridinylmethyl)-N-(5,6,7,8-tetrahydro-8-quinolinyl)-1,4-benzenedimethanamine;
N-[(4-propoxyphenyl)methyl]-N′-(2-pyridinylmethyl)-N-(6,7,8,9-tetrahydro-5H-cyclohepta[b]pyridin-9-yl)-1,4-benzenedimethanamine;
N-(1-phenyl-3,5-dimethylpyrazolin-4-ylmethyl)-N′-(2-pyridinylmethyl)-N-(5,6,7,8-tetrahydro-8-quinolinyl)-1,4-benzenedimethanamine;
N-[1H-imidazol-4-ylmethyl]-N,N′-bis(2-pyridinylmethyl)-1,3-benzenedimethanamine;
N-[(3-methoxy-4,5-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(5,6,7,8-tetrahydro-8-quinolinyl)-1,4-benzenedimethanamine;
N-(5-ethylthiophene-2-ylmethyl)-N′-(2-pyridinylmethyl)-N-(6,7,8,9-tetrahydro-5H-cyclohepta[b]pyridin-9-yl)-1,4-benzenedimethanamine;
N-(5-ethylthiophene-2-ylmethyl)-N′-(2-pyridinylmethyl)-N-(5,6,7,8-tetrahydro-8-quinolinyl)-1,4-benzenedimethanamine;
N-[(2,6-difluorophenyl)methyl]-N′-(2-pyridinylmethyl)-N-(6,7,8,9-tetrahydro-5H-cyclohepta[b]pyridin-9-yl)-1,4-benzenedimethanamine;
N-[(2,6-difluorophenyl)methyl]-N′-(2-pyridinylmethyl)-N-(5,6,7,8-tetrahydro-8-quinolinyl)-1,4-benzenedimethanamine;
N-[(2-difluoromethoxyphenyl)methyl]-N′-(2-pyridinylmethyl)-N-(6,7,8,9-tetrahydro-5H-cyclohepta[b]pyridin-9-yl)-1,4-benzenedimethanamine;
N-(2-difluoromethoxyphenylmethyl)-N′-(2-pyridinylmethyl)-N-(5,6,7,8-tetrahydro-8-quinolinyl)-1,4-benzenedimethanamine;
N-(1,4-benzodioxan-6-ylmethyl)-N′-(2-pyridinylmethyl)-N-(6,7,8,9-tetrahydro-5H-cyclohepta[b]pyridin-9-yl)-1,4-benzenedimethanamine;
N,N′-bis(2-pyridinylmethyl)-N-[1-(N″-phenyl-N″-methylureido)-4-piperidinyl]-1,4-benzenedimethanamine;
N,N′-bis(2-pyridinylmethyl)-N—[N″-p-toluenesulfonylphenylalanyl)-4-piperidinyl]-1,4-benzenedimethanamine;
N-[1-(3-pyridinecarboxamido)-4-piperidinyl]-N,N′-bis(2-pyridinylmethyl)-1,4-benzenedimethanamine;
N-[1-(cyclopropylcarboxamido)-4-piperidinyl]-N,N′-bis(2-pyridinylmethyl)-1,4-benzenedimethanamine;
N-[1-(1-phenylcyclopropylcarboxamido)-4-piperidinyl]-N,N′-bis(2-pyridinylmethyl)-1,4-benzenedimethanamine;
N-(1,4-benzodioxan-6-ylmethyl)-N′-(2-pyridinylmethyl)-N-(5,6,7,8-tetrahydro-8-quinolinyl)-1,4-benzenedimethanamine;
N-[1-[3-(2-chlorophenyl)-5-methyl-isoxazol-4-carboxamido]-4-piperidinyl]-N,N′-bis(2-pyridinylmethyl)-1,4-benzenedimethanamine;
N-[1-(2-thiomethylpyridine-3-carboxamido)-4-piperidinyl]-N,N′-bis(2-pyridinylmethyl)-1,4-benzenedimethanamine;
N-[(2,4-difluorophenyl)methyl]-N′-(2-pyridinylmethyl)-N-(5,6,7,8-tetrahydro-8-quinolinyl)-1,4-benzenedimethanamine;
N-(1-methylpyrrol-2-ylmethyl)-N′-(2-pyridinylmethyl)-N-(5,6,7,8-tetrahydro-8-quinolinyl)-1,4-benzenedimethanamine;
N-[(2-hydroxyphenyl)methyl]-N′-(2-pyridinylmethyl)-N-(5,6,7,8-tetrahydro-8-quinolinyl)-1,4-benzenedimethanamine;
N-[(3-methoxy-4,5-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(5,6,7,8-tetrahydro-8-quinolinyl)-1,4-benzenedimethanamine;
N-[2-(N″-morpholinomethyl)-1-cyclopentyl]-N,N′-bis(2-pyridinylmethyl)-1,4-benzenedimethanamine;
N-[(1-methyl-3-piperidinyl)propyl]-N,N′-bis(2-pyridinylmethyl)-1,4-benzenedimethanamine;
N-(1-methylbenzimidazol-2-ylmethyl)-N′-(2-pyridinylmethyl)-N-(5,6,7,8-tetrahydro-8-quinolinyl)-1,4-benzenedimethanamine;
N-[1-(benzyl)-3-pyrrolidinyl]-N,N′-bis(2-pyridinylmethyl)-1,4-benzenedimethanamine;

N-[[(1-phenyl-3-(N″-morpholino)]propyl]-N,N′-bis(2-pyridinylmethyl)-1,4-benzenedimethanamine;
N-[1-(iso-propyl)-4-piperidinyl]-N,N′-bis(2-pyridinylmethyl)-1,4-benzenedimethanamine;
N-[1-(ethoxycarbonyl)-4-piperidinyl]-N′-(2-pyridinylmethyl)-N-(5,6,7,8-tetrahydro-8-quinolinyl)-1,4-benzenedimethanamine;
N-[(1-methyl-3-pyrazolyl)propyl]-N′-(2-pyridinylmethyl)-N-(5,6,7,8-tetrahydro-8-quinolinyl)-1,4-benzenedimethanamine;
N-[1-methyl-2-(N″,N′-diethylcarboxamido)ethyl]-N,N′-bis(2-pyridinylmethyl)-1,4-benzenedimethanamine;
N-[(1-methyl-2-phenylsulfonyl)ethyl]-N′-(2-pyridinylmethyl)-N-(5,6,7,8-tetrahydro-8-quinolinyl)-1,4-benzenedimethanamine;
N-[(2-chloro-4,5-methylenedioxyphenyl)methyl]-N′-(2-pyridinylmethyl)-N-(5,6,7,8-tetrahydro-8-quinolinyl)-1,4-benzenedimethanamine;
N-[1-methyl-2-[N″-(4-chlorophenyl)carboxamido]ethyl]-N′-(2-pyridinylmethyl)-N-(5,6,7,8-tetrahydro-8-quinolinyl)-1,4-benzenedimethanamine;
N-(1-acetoxyindol-3-ylmethyl)-N′-(2-pyridinylmethyl)-N-(6,7,8,9-tetrahydro-5H-cyclohepta[b]pyridin-9-yl)-1,4-benzenedimethanamine;
N-[(3-benzyloxy-4-methoxyphenyl)methyl]-N′-(2-pyridinylmethyl)-N-(6,7,8,9-tetrahydro-5H-cyclohepta[b]pyridin-9-yl)-1,4-benzenedimethanamine;
N-(3-quinolylmethyl)-N′-(2-pyridinylmethyl)-N-(5,6,7,8-tetrahydro-8-quinolinyl)-1,4-benzenedimethanamine;
N-[(8-hydroxy)-2-quinolylmethyl]-N′-(2-pyridinylmethyl)-N-(6,7,8,9-tetrahydro-5H-cyclohepta[b]pyridin-9-yl)-1,4-benzenedimethanamine;
N-(2-quinolylmethyl)-N′-(2-pyridinylmethyl)-N-(6,7,8,9-tetrahydro-5H-cyclohepta[b]pyridin-9-yl)-1,4-benzenedimethanamine;
N-[(4-acetamidophenyl)methyl]-N′-(2-pyridinylmethyl)-N-(6,7,8,9-tetrahydro-5H-cyclohepta[b]pyridin-9-yl)-1,4-benzenedimethanamine;
N-[1H-imidazol-2-ylmethyl]-N,N′-bis(2-pyridinylmethyl)-1,4-benzenedimethanamine;
N-(3-quinolylmethyl)-N′-(2-pyridinylmethyl)-N-(6,7,8,9-tetrahydro-5H-cyclohepta[b]pyridin-9-yl)-1,4-benzenedimethanamine;
N-(2-thiazolylmethyl)-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(6,7,8,9-tetrahydro-5H-cyclohepta[b]pyridin-9-yl)-1,4-benzenedimethanamine;
N-[(5-benzyloxy)benzo[b]pyrrol-3-ylmethyl]-N,N′-bis(2-pyridinylmethyl)-1,4-benzenedimethanamine;
N-(1-methylpyrazol-2-ylmethyl)-N′-(2-pyridinylmethyl)-N-(6,7,8,9-tetrahydro-5H-cyclohepta[b]pyridin-9-yl)-1,4-benzenedimethanamine;
N-[(4-methyl)-1H-imidazol-5-ylmethyl]-N,N′-bis(2-pyridinylmethyl)-1,4-benzenedimethanamine;

N-[[(4-dimethylamino)-1-naphthalenyl]methyl]-N,N′-bis(2-pyridinylmethyl)-1,4-benzenedimethanamine;
N-[1,5-dimethyl-2-phenyl-3-pyrazolinone-4-ylmethyl]-N,N′-bis(2-pyridinylmethyl)-1,4-benzenedimethanamine;
N-[1-[(1-acetyl-2-(R)-prolinyl]-4-piperidinyl]-N-[2-(2-pyridinyl)ethyl]-N′-(2-pyridinylmethyl)-1,3-benzenedimethanamine;
N-[1-[2-acetamidobenzoyl-4-piperidinyl]-4-piperidinyl]-N-[2-(2-pyridinyl)ethyl]-N′-(2-pyridinylmethyl)-1,3-benzenedimethanamine;
N-[(2-cyano-2-phenyl)ethyl]-N′-(2-pyridinylmethyl)-N-(6,7,8,9-tetrahydro-5H-cyclohepta[b]pyridin-9-yl)-1,4-benzenedimethanamine;

N-[(N″-acetyltryptophanyl)-4-piperidinyl]-N-[2-(2-pyridinyl)ethyl]-N′-(2-pyridinylmethyl)-1,3-benzenedimethanamine;

N—[(N″-benzoylvalinyl)-4-piperidinyl]-N-[2-(2-pyridinyl)ethyl]-N′-(2-pyridinylmethyl)-1,3-benzenedimethanamine;
N-[(4-dimethylaminophenyl)methyl]-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(5,6,7,8-tetrahydro-8-quinolinyl)-1,4-benzenedimethanamine;
N-(1-methylbenzimadazol-2-ylmethyl)-N′-(2-pyridinylmethyl)-N-(6,7,8,9-tetrahydro-5H-cyclohepta[b]pyridin-9-yl)-1,4-benzenedimethanamine;
N-[1-butyl-4-piperidinyl]-N-[2-(2-pyridinyl)ethyl]-N′-(2-pyridinylmethyl)-1,3-benzenedimethanamine;
N-[1-benzoyl-4-piperidinyl]-N-[2-(2-pyridinyl)ethyl]-N′-(2-pyridinylmethyl)-1,3-benzenedimethanamine;
N-[1-(benzyl)-3-pyrrolidinyl]-N-[2-(2-pyridinyl)ethyl]-N′-(2-pyridinylmethyl)-1,3-benzenedimethanamine;
N-[(1-methyl)benzo[b]pyrrol-3-ylmethyl]-N-[2-(2-pyridinyl)ethyl]-N′-(2-pyridinylmethyl)-1,3-benzenedimethanamine;
N-[1H-imidazol-4-ylmethyl]-N-[2-(2-pyridinyl)ethyl]-N′-(2-pyridinylmethyl)-1,3-benzenedimethanamine;
N-[1-(benzyl)-4-piperidinyl]-N-[2-(2-pyridinyl)ethyl]-N′-(2-pyridinylmethyl)-1,4-benzenedimethanamine;
N-[1-methylbenzimidazol-2-ylmethyl]-N-[2-(2-pyridinyl)ethyl]-N′-(2-pyridinylmethyl)-1,4-benzenedimethanamine;
N-[(2-phenyl)benzo[b]pyrrol-3-ylmethyl]-N-[2-(2-pyridinyl)ethyl]-N′-(2-pyridinylmethyl)-1,4-benzenedimethanamine;
N-[(6-methylpyridin-2-yl)methyl]-N′-(2-pyridinylmethyl)-N-(5,6,7,8-tetrahydro-8-quinolinyl)-1,4-benzenedimethanamine;
N-(3-methyl-1H-pyrazol-5-ylmethyl)-N′-(2-pyridinylmethyl)-N-(5,6,7,8-tetrahydro-8-quinolinyl)-1,3-benzenedimethanamine;
N-[(2-methoxyphenyl)methyl]-N′-(2-pyridinylmethyl)-N-(5,6,7,8-tetrahydro-8-quinolinyl)-1,3-benzenedimethanamine;
N-[(2-ethoxyphenyl)methyl]-N′-(2-pyridinylmethyl)-N-(6,7,8,9-tetrahydro-5H-cyclohepta[b]pyridin-9-yl)-1,3-benzenedimethanamine;
N-(benzyloxyethyl)-N′-(2-pyridinylmethyl)-N-(5,6,7,8-tetrahydro-8-quinolinyl)-1,3-benzenedimethanamine;
N-[(2-ethoxy-1-naphthalenyl)methyl]-N′-(2-pyridinylmethyl)-N-(5,6,7,8-tetrahydro-8-quinolinyl)-1,3-benzenedimethanamine;
N-[(6-methylpyridin-2-yl)methyl]-N′-(2-pyridinylmethyl)-N-(5,6,7,8-tetrahydro-8-quinolinyl)-1,3-benzenedimethanamine;
1-[[4-[[(2-pyridinylmethyl)amino]methyl]phenyl]methyl]guanidine;
N-(2-pyridinylmethyl)-N-(8-methyl-8-azabicyclo[3.2.1]octan-3-yl)-1,4-benzenedimethanamine;
1-[[4-[[(2-pyridinylmethyl)amino]methyl]phenyl]methyl]homopiperazine;
1-[[3-[[(2-pyridinylmethyl)amino]methyl]phenyl]methyl]homopiperazine;
trans and cis-1-[[4-[[(2-pyridinylmethyl)amino]methyl]phenyl]methyl]-3,5-piperidinediamine;
N,N′-[1,4-Phenylenebis(methylene)]bis-4-(2-pyrimidyl)piperazine;
1-[[4-[[(2-pyridinylmethyl)amino]methyl]phenyl]methyl]-1-(2-pyridinyl)methylamine;
2-(2-pyridinyl)-5-[[(2-pyridinylmethyl)amino]methyl]-1,2,3,4-tetrahydroisoquinoline;
1-[[4-[[(2-pyridinylmethyl)amino]methyl]phenyl]methyl]-3,4-diaminopyrrolidine;
1-[[4-[[(2-pyridinylmethyl)amino]methyl]phenyl]methyl]-3,4-diacetylaminopyrrolidine;
8-[[4-[[(2-pyridinylmethyl)amino]methyl]phenyl]methyl]-2,5,8-triaza-3-oxabicyclo[4.3.0]nonane; and
8-[[4-[[(2-pyridinylmethyl)amino]methyl]phenyl]methyl]-2,5,8-triazabicyclo[4.3.0]nonane.

### APPENDIX B

Exemplary CXCR₄ antagonists include compounds of formula (1A):

V—CR₂—Ar¹—CR₂NR—(CR₂)ₓ—Ar (1A)

wherein V is a substituted heterocycle of 9-24 members containing 2-4 optionally substituted amine nitrogen atoms spaced from each other by 2 or more optionally substituted carbon atoms, and which heterocycle may optionally comprise a fused aromatic or heteroaromatic ring, and wherein

(a) said heterocycle contains at least one O or S, said O or S spaced from any adjacent heteroatom by at least 2 carbon atoms, and wherein said S is optionally oxidized or

(b) at least one carbon atom in said ring is substituted by an electron-withdrawing substituent, or

(c) both (a) and (b);

and wherein each R is independently H or a straight chain, branched or cyclic alkyl containing 1-6C;

x is 0-4;

Ar¹ is an unsubstituted or substituted aromatic or heteroaromatic moiety; and

Ar² is an unsubstituted or substituted aromatic or heterocyclic group.

In the above Formula (1A), V may contain 2-4 N, preferably 3-4 N if there is no additional heteroatom. Preferable ring sizes for V are 9-18 members, more preferably 12-16 members. V may also include a fused aromatic or heteroaromatic ring, preferably 1,2 or 1,3 or 1,4 phenylene or 2,6 or 2,5 or 2,4 or 2,3 pyridinylene. The fused ring may also be, for example, 2,5 or 2,6 pyrimidinylene or 2,4 or 2,3 pyrrolylene.

In the above Formula 1A, the electron withdrawing substituents present at least one C in ring V may be halogen, nitro, cyano, carboxylic acid, a carboxylic ester formed from an alcohol of 1-6C, an amide formed from an amine of 0-12C, a sulfonic or sulfinic acid, ester or amide, CF₃, and the like. A preferred electron withdrawing substituent is ═O, as well as halo. Examples of halogen include fluorine, chlorine, bromine, iodine, with fluorine and chlorine preferred.

In the above Formula (1A), Ar² may be an optionally substituted heterocyclic group or aromatic group. Examples of aromatic groups include but are not limited to benzene, naphthalene, dihydronaphthalene and tetrahydronaphthalene. Examples of heterocyclic groups include 5 to 6-membered saturated, partially saturated, or aromatic heterocyclic rings containing 1 to 4 heteroatoms selected from nitrogen, oxygen and sulfur. The heterocycles may be pyridine, quinoline, isoquinoline, imidazole, benzimidazole, azabenzimidazole, benzotriazole, furan, benzofuran, thiazole, benzothiazole, oxazole, benzoxazole, pyrrole, indole, indoline, indazole, pyrrolidine, pyrrolidone, pyrroline, piperidine, piperazine, tetrahydroquinoline, tetrahydroisoquinoline, pyrazole, thiophene, isoxazole, isothiazole, triazole, tetrazole, oxadiazole, thiadiazole, morpholine, thiamorpholine, pyrazolidine, imidazolidine, imidazoline, tetrahydropyran, dihydropyran, benzopyran, dioxane, dithiane, tetrahydrofuran, tetrahydrothiophene, dihydrofuran, dihydrothiophene, and the like. Oxides of the nitrogen and sulfur containing heterocycles are also included.

The optional substituents on Ar² include alkyl (1-6C), alkenyl (1-6C), alkynyl (1-6C), halo, nitro, cyano, carboxylic acid, carboxylic ester formed from an alcohol with 1-6C, an amide formed from an amine of 0-12C, a sulfonic or sulfinic acid, ester or amide, OR, SR, NR₂, OCR, OOCR, NRCOR, all wherein R is hydrogen or straight or branched chain alkyl (1-6C), an optionally substituted aromatic or heterocyclic group, CF₃, and the like. Preferred substituents include alkyl, OR, NR₂, and halo. Preferred embodiments of Ar include phenyl, pyridinyl, pyrimidinyl and imidazolyl.

In the above Formula (1A), Ar¹ may be a 5-6 membered aromatic system which is bivalent benzene, pyridine, thiophene, pyrimidine, and the like. Ar¹ may optionally be substituted by alkyl, alkenyl, halo, nitro, cyano, CF₃, COOR, CONR₂, OCR, OOCR, NRCOR, OR, NR₂, SR (where R is H or alkyl 1-6C), sulfonic or sulfinic acids, esters or amides and the like. Preferred embodiments of Ar¹ are phenylene, especially 1,3 and 1,4 phenylene and pyridinylene, preferably 2,6 pyridinylene, and 3,5 pyridinylene.

Further, in the compounds of Formula (1A), each R group may be hydrogen or alkyl of 1-2C, preferably hydrogen. The R group may be coupled to a nitrogen is hydrogen or alkyl 1-6C, preferably straight chain alkyl 1-3C, more preferably H or methyl. In one example, 1, 2, 3, 4, or 5 of the R groups are methyl or ethyl and the remaining R groups are hydrogen.

In one embodiment, the CXCR4 antagonist has formula

V—CH₂—Ar¹—CH₂NR—CH₂—Ar

wherein V is a heterocycle as defined in formula (1A), and wherein:

(a) said heterocycle is substituted with halo or ═O; or

(b) said heterocycle contains O or S; or

(c) both (a) and (b),

and wherein Ar¹ is unsubstituted 1, 3 or 1,4-phenylene, R is H, methyl or ethyl and Ar² is unsubstituted phenyl or pyridinyl. Preferred embodiments of x are 0-2 and 1-2.

The heterocycle V may contain 3 N and at least one carbon atom in the heterocycle that is substituted by at least one fluoro substituent. The R moiety may independently be hydrogen or methyl. The number of (CR₂)ₓ groups may be 0-4, 0-2, or 1-2. The Ar¹ moiety may be 1, 3 or 1,4-phenylene. The Ar² moiety may be phenyl or pyridyl. The heterocycle V may be a 12-16 membered heterocycle, or may contain O or S as a ring member. The heterocycle V may also contain an oxidized sulfur as a ring member. In one example, at least one carbon in the heterocycle V is substituted by ═O.

Compounds of formula (1A), and methods of synthesizing such compounds are described in WO 01/44229, incorporated herein by reference. Examples of compounds of Formula (1A), its pharmaceutically acceptable salts or metal complexes thereof, include but are not limited to:
N-[4-(11-fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-onyl)-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,4,7-triazacyclotetradecanyl)-1,4-phenylenebis(methylene)]-2-(aminomethyl)pyridine;
N-[4-(11-thia-1,4,7-triazacyclotetradecanyl)-1,4-phenylenebis(methylene)]-2-(aminomethyl)pyridine;
N-[4-(11-sulfoxo-1,4,7-triazacyclotetradecanyl)-1,4-phenylenebis(methylene)]-2-(aminomethyl)pyridine;
N-[4-(11-sulfono-1,4,7-triazacyclotetradecanyl)-1,4-phenylenebis(methylene)]-2-(aminomethyl)pyridine; or
N-[4-(3-carboxo-1,4,7-triazacyclotetradecanyl)-1,4-phenylenebis(methylene)]-2-(aminomethyl)pyridine.

In another aspect, the CXCR4 compound for use in the methods of the present invention is exemplified by compounds having formula (1B):

V—CR¹R²—Ar—CR³R⁴—N(R⁵)—(CR⁶R⁷)ₓ—R⁸ (1B)

wherein V is an optionally substituted 1,4,8,11-tetraazacyclotetradecanyl, 4,7,10,17-tetraazabicyclo[13.3.1]heptadeca-1(17),13,15-trienyl, 1,4,7-triazacyclotetradecanyl, 4,7,10-triazabicyclo[13.3.1]heptadeca-1(17),13,15-trienyl, 1,7-diazacyclotetradecanyl, or 4,10-diazabicyclo[13.31.1]heptadeca-1(17),13,15-trienyl system;

R¹ to R⁷ may be the same or different and are independently selected from hydrogen or straight, branched or cyclic C₁₋₆ alkyl;

R⁸ is pyridyl, pyrimidinyl, pyrazinyl, imidazolyl, thiophene-yl, thiophenyl, aminobenzyl, piperidinyl, purine, piperazinyl, phenylpiperazinyl, or mercaptan;

Ar is a phenylene ring optionally substituted at single or multiple positions with alkyl, aryl, amino, alkoxy, hydroxy, halogen, carboxyl and/or carboxamido; and

x is 1 or 2.

In the above formula (1B), the V moiety may be optionally substituted by hydroxyl, alkoxy, thiol, thioalkyl, halogen, nitro, carboxy, amido, sulfonic acid, and/or phosphate.

Compounds of Formula (1B), its pharmaceutically acceptable salts or metal complexes thereof, and methods of synthesizing such compounds are described in WO 00/02870, which is incorporated herein by reference. Examples of compounds having formula (1B) include but are not limited to:
N-[1,4,8,11-tetraazacyclotetra-decanyl-1,4-phenylenebis-(methylene)]-2-(aminomethyl)pyridine;
N-[1,4,8,11-tetraazacyclotetra-decanyl-1,4-phenylenebis(methylene)]-N-methyl-2-(aminomethyl)pyridine;
N-[1,4,8,11-tetraazacyclotetra-decanyl-1,4-phenylenebis(methylene)]-4-(aminomethyl)pyridine;
N-[1,4,8,11-tetraazacyclotetra-decanyl-1,4-phenylenebis(methylene)]-3-(aminomethyl)pyridine;
N-[1,4,8,11-tetraazacyclotetra-decanyl-1,4-phenylenebis(methylene)]-(2-aminomethyl-5-methyl)pyrazine;
N-[1,4,8,11-tetraazacyclotetra-decanyl-1,4-phenylenebis(methylene)]-2-(aminoethyl)pyridine;
N-[1,4,8,11-tetraazacyclotetra-decanyl-1,4-phenylenebis(methylene)]-2-(aminomethyl)thiophene;
N-[1,4,8,11-tetraazacyclotetra-decanyl-1,4-phenylenebis(methylene)]-2-(aminomethyl)mercaptan;
N-[1,4,8,11-tetraazacyclotetra-decanyl-1,4-phenylenebis(methylene)]-2-amino benzylamine;
N-[1,4,8,11-tetraazacyclotetra-decanyl-1,4-phenylenebis(methylene)]-4-amino benzylamine;
N-[1,4,8,11-tetraazacyclotetra-decanyl-1,4-phenylenebis(methylene)]-4-(aminoethyl)imidazole;
N-[1,4,8,11-tetraazacyclotetra-decanyl-1,4-phenylenebis(methylene)]-benzylamine;
N-[4-(1,4,7-triazacyclotetra-decanyl)-1,4-phenylenebis(methylene)]-2-(aminomethyl)pyridine;
N-[7-(4,7,10,17-tetraazabicyclo[13.3.1]heptadeca-1(17),13,15-trienyl)-1,4-phenylenebis(methylene)]-2-(aminomethyl)pyridine;
N-[7-(4,7,10-triazabicyclo[13.3.1]heptadeca-1(17),13,15-trienyl)-1,4-phenylenebis(methylene)]-2-(aminomethyl)pyridine;
N-[1-(1,4,7-triazacyclotetra-decanyl)-1,4-phenylenebis(methylene)]-2-(aminomethyl)pyridine;
N-[4-[4,7,10,17-tetraazabicyclo[13.3.1]heptadeca-1(17),13,15-trienyl]-1,4-phenylenebis(methylene)]-2-(aminomethyl)pyridine;
N-[4-[4,7,10-triazabicyclo[13.3.1]heptadeca-1(17),13,15-trienyl]-1,4-phenylenebis(methylene)]-2-(aminomethyl)pyridine;
N-[1,4,8,11-tetraazacyclotetradecanyl-1,4-phenylenebis(methylene)]-purine;
1-[1,4,8,11-tetraazacyclotetradecanyl-1,4-phenylenebix(methylene)]-4-phenylpiperazine;
N-[4-(1,7-diazacyclotetradecanyl)-1,4-phenylenebis(methylene)]-2-(aminomethyl)pyridine; and
N-[7-(4,10-diazabicyclo[13.3.1]heptadeca-1(17),13,15-trienyl)-1,4-phenylenebis(methylene)]-2-(aminomethyl)pyridine.

Other CXCR₄ inhibitors are of formula (1C):

V²—CR₉R₁₀—Ar² (1C)

wherein V² is an optionally substituted 1,4,8,11-tetraazacyclotetra-decanyl or 4,7,10,17-tetraazabicyclo[13.3.1]heptadeca-1(17),13,15-trienyl system;

R₉ and R₁₀ may be the same or different and are independently selected from hydrogen or straight, branched or cyclic C₁₋₆ alkyl;

Ar₂ is an aromatic or heterocyclic ring each optionally substituted at single or multiple positions with electron-donating or withdrawing groups and/or aromatic and heterocyclic groups and their alkyl derivatives thereof, and the acid addition salts and metal complexes.

In the above Formula (1C), Ar₂ may be optionally substituted with alkyl, aryl, amino, alkoxy, hydroxy, halogen, carboxyl and/or carboxamido. In particular examples, Ar₂ is optionally substituted with alkoxy, alkyl, or halogen.

Compounds having formula (1C), and methods of synthesizing the same, are described in WO 00/02870, incorporated herein by reference. Examples of compounds having formula (1C) include but are not limited to:
1-[2,6-dimethoxypyrid-4-yl(methylene)]-1,4,8,11-tetraazacyclotetradecane;
1-[2-chloropyrid-4-yl(methylene)]-1,4,8,11-tetraazacyclotetradecane;
1-[2,6-dimethylpyrid-4-yl(methylene)]-1,4,8,11-tetraazacyclotetradecane;
1-[2-methylpyrid-4-yl(methylene)]-1,4,8,11-tetraazacyclotetradecane;
1-[2,6-dichloropyrid-4-yl(methylene)]-1,4,8,11-tetraazacyclotetradecane;
1-[2-chloropyrid-5-yl(methylene)]-1,4,8,11-tetraazacyclotetradecane; and
7-[4-methylphenyl (methylene)]-4,7,10,17-tetraazabicyclo[13.3.1]heptadeca-1(17),13,15-triene.

Other CXCR4 antagonists are of formula (1D):

V—R-A-R′—W (1D)

wherein V and W are independently cyclic polyamine moieties having from 9 to 32 ring members and from 3 to 8 amine nitrogens in the ring spaced by 2 or more carbon atoms from each other, and having one or more aromatic or heteroaromatic rings fused thereto,

A is an aromatic or heteroaromatic moiety when V and W have one or more aromatic or heteroaromatic moieties fused thereto, with or without an additional heteroatom other than nitrogen incorporated in the ring, or A is an aromatic or heteroaromatic moiety when V and W contain a heteroatom other than nitrogen incorporated in the ring without having one or more aromatic or heteroaromatic moieties fused thereto,

and R and R′ are each a substituted or unsubstituted alkylene chain or heteroatom-containing chain which spaces the cyclic polyamines and the moiety A.

In the above Formula (1D), R and R′ may each be methylene. In one example, A is 1,3- or 1,4-phenylene. In another example, each V and W is an unsubstituted or substituted tricyclic or bicyclic ring system containing only carbon and nitrogen atoms in the rings. One of the cyclic ring systems may be a 10 to 20 membered polyamine ring system having from 3 to 6 amine nitrogen atoms, and the ring system or systems is a fused benzyl or pyridinyl ring system.

Compounds having formula (1D), and methods of synthesizing such compounds, are described in U.S. Pat. No. 5,698,546, incorporated herein by reference. These compounds include but are not limited to:
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo
[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-[(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene; and
17,17′-[1,4-phenylene-bis(methylene)]bis-3,6,14,17,23,24-hexaazatricyclo[17.3.1.1^{8,12}]tetracosa-1(23),8,10,12(24),19,21-hexaene.

Other CXCR4 antagonists are of formula (1E):

Z-R-A-R′-Y (1E)

where Z and Y are identical cyclic polyamine moieties having from 10 to 15 ring members and from 3 to 6 amine nitrogens in the ring spaced by 2 or more carbon atoms from each other, said amine nitrogens being the only ring heteroatoms,

A is an aromatic or heteroaromatic moiety other than quinoline,

R and R′ are each methylene linked to nitrogen atoms in Z and Y, the amine nitrogen atoms being otherwise unsubstituted.

In the above formula (1E), each moiety Z and Y may have 14 ring members and 4 amine nitrogens in the ring. Compounds having formula (1E), and methods of synthesizing such compounds, are described in U.S. Pat. No. 5,583,131, incorporated herein by reference. These compounds include but are not limited to:
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane (AMD 3100);
1,1′-[1,4-phenylene-bis-(methylene)]-bis-1,4,8,11-tetraazacyclotetradecane;
bis-zinc or bis-copper complex of 1,1′-[1,4-phenylene-bis-(methylene)]-bis-1,4,8,11-tetraazacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis-(methylene)]-1,4,8,11-tetraazacyclotetradecane-1,4,7,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1′-[5-nitro-1,3-phenylenebis(methylene)]bis-1,4,8,11-tetraazacyclotetradecane;
1,1′-[2,4,5,6-tetrachloro-1,3-phenylenebis(methylene)]bis-1,4,8,11-tetraazacyclotetradecane;
1,1′-[2,3,5,6-tetra-fluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane; and
1,1′-[6-phenyl-2,4-pyridinebis-(methylene)]-bis-1,4,8,11-tetraazacyclotetradecane.

The CXCR4 antagonist may be of formula (1F):

Z-(A)ₙ-Y (1F)

where Z and Y are independently cyclic polyamine moieties having from 9 to 32 ring members and from 3 to 8 amine nitrogen atoms in the ring,

A is a linking atom or group, and n is 0 or an integer from 1 to 6.

In the above formula (1F) each Z and Y moiety may have 10 to 24 ring members, or 12 to 18 ring members. Each Z and Y moiety may also have 4 to 6 amine nitrogen atoms in the ring. In one example, n is 0. In another example, A is methylene.

Compounds having formula (1F), and methods of synthesizing such compounds, are described in U.S. Pat. No. 5,021,409, incorporated herein by reference. These compounds include but are not limited to:
2,2′-bicyclam, 6,6′-bicyclam;
3,3′-(bis-1,5,9,13-tetraaza cyclohexadecane);
3,3′-(bis-1,5,8,11,14-pentaazacyclohexadecane);
methylene (or polymethylene) di-1-N-1,4,8,11-tetraaza cyclotetradecane;
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane; and
11,11′-(1,2-hexanediyl)bis-1,4,8,11-tetraazacyclotetradecane.

Other CXCR4 antagonists are of formula (2A):

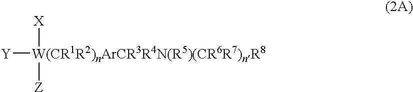

W is a nitrogen atom and Y is void, or W is a carbon atom and Y═H;

R¹ to R⁷ may be the same or different and are independently hydrogen or straight, branched or cyclic C₁₋₆ alkyl;

R⁸ is an optionally substituted heterocyclic group or an optionally substituted aromatic group

Ar is an aromatic or heteroaromatic ring optionally substituted at single or multiple, non-linking positions with electron-donating or withdrawing groups;

n and n′ are independently, 0-2;

X is a group of the formula:

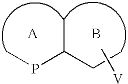

wherein, Ring A is an optionally substituted, saturated or unsaturated 5 or 6-membered ring, and P is an optionally substituted nitrogen atom and wherein any heteroatom in addition to P in ring A is N;

wherein Ring B is an optionally substituted 5 to 7-membered ring;

wherein Ring A or Ring B is bound to group W from any position through group V;

wherein V is a chemical bond or V is a (CH₂), group (where n″=1-2), or V is a C═O group; and

wherein Z is selected from the group consisting of: a hydrogen atom; an optionally substituted C₁₋₆ alkyl group; an optionally substituted aromatic or heterocyclic group; an optionally substituted amino group; an optionally substituted C₁₋₆ alkylamino or C₃₋₇ cycloalkylamino group; and a substituted carbonyl group; or

the pharmaceutically acceptable acid addition salts thereof;

wherein said compound may be in any stereoisomeric form or present as a mixture of stereoisomeric forms thereof;

wherein Ring B is selected from the group consisting of: benzene and a 5 to 7-membered cycloalkyl ring; and the optionally substituted forms thereof.

In the above formula (2A), Ring A may be pyridine; pyrimidine; pyrazine; pyridazine; triazine; piperidine; piperazine; imidazole; pyrazole; or triazole. and the optionally substituted forms thereof. Ring B may be cyclopentyl; cyclohexyl; cycloheptyl; cyclopentenyl; cyclohexenyl; or cycloheptenyl, and the optionally substituted forms thereof. In one embodiment, Ring A and Ring B together are optionally substituted dihydroquinoline or tetrahydroquinoline.

In the above formula (2A), Ring A and Ring B are independently optionally substituted with a substituent selected from the group consisting of: halogen; nitro; cyano; carboxylic acid; an optionally substituted alkyl, alkenyl or cycloalkyl group; an optionally substituted hydroxyl group; an optionally substituted thiol group; an optionally substituted amino or acyl group; an optionally substituted carboxylate, carboxamide or sulfonamide group; and an optionally substituted aromatic or heterocyclic group. In one embodiment, the optional substituent in Ring A or Ring B is independently an optionally substituted aralkyl or heterocycloalkyl, wherein said heterocycloalkyl is a 5 or 6 membered ring containing 1-4 heteroatoms. For example, the optionally substituted aralkyl or heterocycloalkyl may be phenylC₁₋₄alkyl; phenylmethyl (benzyl); phenethyl; pyridinylmethyl; or pyridinylethyl.

In the above formula (2A), Z may be an optionally substituted C₁₋₆alkyl group, wherein said C₁₋₆alkyl group is substituted with one or more substituents selected from the group consisting of: halogen; nitro; cyano; carboxylic acid; an optionally substituted alkyl, alkenyl or cycloalkyl group; an optionally substituted hydroxyl group; an optionally substituted thiol group; an optionally substituted amino or acyl group; an optionally substituted carboxylate, carboxamide or sulfonamide group; and an optionally substituted aromatic or heterocyclic group.

In the above formula (2A), Z is an optionally substituted aromatic or heterocyclic group or a C₁₋₆alkyl group optionally substituted with an optionally substituted aromatic or heterocyclic group. In one embodiment, Z is a C₁₋₆ alkyl group substituted with an optionally substituted aromatic or heterocyclic group. The optionally substituted aromatic group may be substituted with a substituent selected from the group consisting of: benzene; naphthalene; dihydronaphthalene; and tetrahydronaphthalene; and wherein said optionally substituted heterocyclic group is a 5 to 6-membered saturated, partially saturated, or aromatic heterocyclic ring containing 1 to 4 heteroatoms selected from nitrogen, oxygen and sulfur. The heterocyclic group selected from the group consisting of: pyridine, quinoline, isoquinoline, imidazole, benzimidazole, azabenzimidazole, benzotriazole, furan, benzofuran, thiazole, benzothiazole, oxazole, benzoxazole, pyrrole, indole, indoline, indazole, pyrrolidine, pyrrolidone, pyrroline, piperidine, piperazine, tetrahydroquinoline, tetrahydroisoquinoline, pyrazole, thiophene, isoxazole, isothiazole, triazole, tetrazole, oxadiazole, thiadiazole, morpholine, thiamorpholine, pyrazolidine, imidazolidine, imidazoline, tetrahydropyran, dihydropyran, benzopyran, dioxane, dithiane, tetrahydrofuran, tetrahydrothiophene, dihydrofuran, and dihydrothiophene. The heterocyclic group may also contain nitrogen or sulfur heteroatoms; and wherein said nitrogen or sulfur heteroatoms are optionally in the form of oxides.

The CXCR4 antagonists also include compounds of formula (2B):

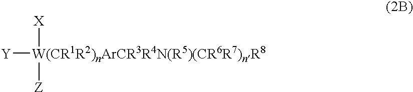

wherein, W is a nitrogen atom and Y is void;

R¹ to R⁷ may be the same or different and are independently hydrogen or straight, branched or cyclic C₁₋₆ alkyl;

R⁸ is an optionally substituted heterocyclic group or an optionally substituted aromatic group

Ar is an aromatic or heteroaromatic ring optionally substituted at single or multiple, non-linking positions with electron-donating or withdrawing groups;

n and n′ are independently, 0-2;

X is a group of the formula:

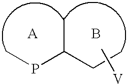

wherein, Ring A is an optionally substituted, saturated or unsaturated 5 or 6-membered ring, and P is an optionally substituted nitrogen atom and wherein any heteroatom in ring A or B is N;

wherein Ring B is an optionally substituted 5 to 7-membered ring;

wherein Ring A or Ring B is bound to group W from any position through group V;

wherein V is a chemical bond or V is a (CH₂)_{n″} group (where n″=1-2), or V is a C═O group; and

wherein Z is selected from the group consisting of: a hydrogen atom; an optionally substituted C₁₋₆ alkyl group; an optionally substituted aromatic or heterocyclic group; an optionally substituted amino group; an optionally substituted C₁₋₆ alkylamino or C₃₋₇ cycloalkylamino group; and a substituted carbonyl group; or the pharmaceutically acceptable acid addition salts thereof;

wherein said compound may be in any stereoisomeric form or present as a mixture of stereoisomeric forms thereof.

In the above formula (2B), Ring A may be pyridine; pyrimidine; pyrazine; pyridazine; triazine; piperidine; piperazine; imidazole; pyrazole; or triazole, and the optionally substituted forms thereof. Ring B may be benzene or a 5 to 7-membered cycloalkyl ring; and the optionally substituted forms thereof. For example, Ring B may be cyclopentyl; cyclohexyl; cycloheptyl; cyclopentenyl; cyclohexenyl; or cycloheptenyl. and the optionally substituted forms thereof.

In the above formula (2B), Ring A and Ring B together may be an optionally substituted dihydroquinoline or tetrahydroquinoline. For example, Ring A and Ring B are independently optionally substituted with a substituent selected from the group consisting of: halogen; nitro; cyano; carboxylic acid; an optionally substituted alkyl, alkenyl or cycloalkyl group; an optionally substituted hydroxyl group; an optionally substituted thiol group; an optionally substituted amino or acyl group; an optionally substituted carboxylate, carboxamide or sulfonamide group; and an optionally substituted aromatic or heterocyclic group. In one example, the optional substituent in Ring A or Ring B is independently an optionally substituted aralkyl or heterocycloalkyl, wherein said heterocycloalkyl is a 5 or 6 membered ring containing 1-4 heteroatoms. The optionally substituted aralkyl or heterocycloalkyl is selected from the group consisting of: phenylC₁₋₄alkyl; phenylmethyl (benzyl); phenethyl; pyridinylmethyl; and pyridinylethyl.

In the above formula (2B), Z may be an optionally substituted C₁₋₆alkyl group, wherein said C₁₋₆alkyl group is substituted with one or more substituents selected from the group consisting of: halogen; nitro; cyano; carboxylic acid; an optionally substituted alkyl, alkenyl or cycloalkyl group; an optionally substituted hydroxyl group; an optionally substituted thiol group; an optionally substituted amino or acyl group; an optionally substituted carboxylate, carboxamide or sulfonamide group; and an optionally substituted aromatic or heterocyclic group. In one example, Z is a C₁₋₆ alkyl group substituted with an optionally substituted aromatic or heterocyclic group.

In another example, Z is an optionally substituted aromatic or heterocyclic group or a C₁₋₆alkyl group optionally substituted with an optionally substituted aromatic or heterocyclic group. For example, the optionally substituted aromatic group is substituted with a substituent selected from the group consisting of: benzene; naphthalene; dihydronaphthalene; and tetrahydronaphthalene; and wherein said optionally substituted heterocyclic group is a 5 to 6-membered saturated, partially saturated, or aromatic heterocyclic ring containing 1 to 4 heteroatoms selected from nitrogen, oxygen and sulfur. The heterocyclic group may be pyridine, quinoline, isoquinoline, imidazole, benzimidazole, azabenzimidazole, benzotriazole, furan, benzofuran, thiazole, benzothiazole, oxazole, benzoxazole, pyrrole, indole, indoline, indazole, pyrrolidine, pyrrolidone, pyrroline, piperidine, piperazine, tetrahydroquinoline, tetrahydroisoquinoline, pyrazole, thiophene, isoxazole, isothiazole, triazole, tetrazole, oxadiazole, thiadiazole, morpholine, thiamorpholine, pyrazolidine, imidazolidine, imidazoline, tetrahydropyran, dihydropyran, benzopyran, dioxane, dithiane, tetrahydrofuran, tetrahydrothiophene, dihydrofuran, or dihydrothiophene. In other examples, the heterocyclic group contains nitrogen or sulfur heteroatoms; and wherein said nitrogen or sulfur heteroatoms are optionally in the form of oxides.

In one embodiment, the CXCR4 antagonist is a compound selected from the group consisting of:
N-(2-pyridinylmethyl)-N′-(6,7,8,9-tetrahydro-5H-cyclohepta[b]pyridin-9-yl)-1,4-benzenedimethanamine;
N-(2-pyridinylmethyl)-N′-(5,6,7,8-tetrahydro-8-quinolinyl)-1,4-benzenedimethanamine;
N-(2-pyridinylmethyl)-N′-(6,7-dihydro-5H-cyclopenta[b]pyridin-7-yl)-1,4-benzenedimethanamine;
N-(2-pyridinylmethyl)-N′-(1,2,3,4-tetrahydro-1-naphthalenyl)-1,4-benzenedimethanamine;
N-(2-pyridinylmethyl)-N′-(1-naphthalenyl)-1,4-benzenedimethanamine;
N-(2-pyridinylmethyl)-N′-(8-quinolinyl)-1,4-benzenedimethanamine;
N-(2-pyridinylmethyl)-N′-[2-[(2-pyridinylmethyl)amino]ethyl]-N′-(1-methyl-1,2,3,4-tetrahydro-8-quinolinyl)-1,4-benzenedimethanamine;
N-(2-pyridinylmethyl)-N′-[2-[(1H-imidazol-2-ylmethyl)amino]ethyl]-N′-(1-methy 1-1,2,3,4-tetrahydro-8-quinolinyl)-1,4-benzenedimethanamine;
N-(2-pyridinylmethyl)-N′-(1,2,3,4-tetrahydro-8-quinolinyl)-1,4-benzenedimethanamine;
N-(2-pyridinylmethyl)-N′-[2-[(1H-imidazol-2-ylmethyl)amino]ethyl]-N′-(1,2,3,4-tetrahydro-1-naphthalenyl)-1,4-benzenedimethanamine;
N-(2-pyridinylmethyl)-N′-(2-phenyl-5,6,7,8-tetrahydro-8-quinolinyl)-1,4-benzenedimethanamine;
N,N′-bis(2-pyridinylmethyl)-N′-(2-phenyl-5,6,7,8-tetrahydro-8-quinolinyl)-1,4-benzenedimethanamine;
N-(2-pyridinylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-8-quinolinyl)-1,4-benzenedimethanamine;
N-(2-pyridinylmethyl)-N′-[(2-amino-3-phenyl)propyl]-N′-(5,6,7,8-tetrahydro-8-quinolinyl)-1,4-benzenedimethanamine;
N-(2-pyridinylmethyl)-N′-(1H-imidazol-4-ylmethyl)-N′-(5,6,7,8-tetrahydro-8-quinolinyl)-1,4-benzenedimethanamine;
N-(2-pyridinylmethyl)-N′-(2-quinolinylmethyl)-N′-(5,6,7,8-tetrahydro-8-quinolin yl)-1,4-benzenedimethanamine;
N-(2-pyridinylmethyl)-N′-(2-(2-naphthoyl)aminoethyl)-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[3-((2-naphthalenylmethyl)amino)propyl]-N′-(5,6,7,8-tetrahydro-8-quinolinyl)-1,4-benzenedimethanamine;
N-(2-pyridinylmethyl)-N′-[2-(S)-pyrollidinylmethyl]-N′-(5,6,7,8-tetrahydro-8-quinolinyl)-1,4-benzenedimethanamine;
N-(2-pyridinylmethyl)-N′-[2-(R)-pyrollidinylmethyl]-N′-(5,6,7,8-tetrahydro-8-quinolinyl)-1,4-benzenedimethanamine;
N-(2-pyridinylmethyl)-N′-[3-pyrazolylmethyl]-N′-(5,6,7,8-tetrahydro-8-quinolinyl)-1,4-benzenedimethanamine;
N-(2-pyridinylmethyl)-N′-[2-pyrrolylmethyl]-N′-(5,6,7,8-tetrahydro-8-quinolinyl)-1,4-benzenedimethanamine;
N-(2-pyridinylmethyl)-N′-[2-thiopheneylmethyl]-N′-(5,6,7,8-tetrahydro-8-quinolinyl)-1,4-benzenedimethanamine

N-(2-pyridinylmethyl)-N′-[2-thiazolylmethyl]-N′-(5,6,7,8-tetrahydro-8-quinolinyl)-1,4-benzenedimethanamine;
N-(2-pyridinylmethyl)-N′-[2-furanylmethyl]-N′-(5,6,7,8-tetrahydro-8-quinolinyl)-1,4-benzenedimethanamine;
N-(2-pyridinylmethyl)-N′-[2-[(phenylmethyl)amino]ethyl]-N′-(5,6,7,8-tetrahydro-8-quinolinyl)-1,4-benzenedimethanamine;
N-(2-pyridinylmethyl)-N′-(2-aminoethyl)-N′-(5,6,7,8-tetrahydro-8-quinolinyl)-1,4-benzenedimethanamine;
N-(2-pyridinylmethyl)-N′-3-pyrrolidinyl-N′-(5,6,7,8-tetrahydro-8-quinolinyl)-1,4-benzenedimethanamine
N-(2-pyridinylmethyl)-N′-4-piperidinyl-N′-(5,6,7,8-tetrahydro-8-quinolinyl)-1,4-benzenedimethanamine;
N-(2-pyridinylmethyl)-N′-[2-[(phenyl)amino]ethyl]-N′-(5,6,7,8-tetrahydro-8-quinolinyl)-1,4-benzenedimethanamine;
N-(2-pyridinylmethyl)-N′-(7-methoxy-1,2,3,4-tetrahydro-2-naphthalenyl)-1,4-benzenedimethanamine;
N-(2-pyridinylmethyl)-N′-(6-methoxy-1,2,3,4-tetrahydro-2-naphthalenyl)-1,4-benzenedimethanamine;
N-(2-pyridinylmethyl)-N′-(1-methyl-1,2,3,4-tetrahydro-2-naphthalenyl)-1,4-benzenedimethanamine;
N-(2-pyridinylmethyl)-N′-(7-methoxy-3,4-dihydronaphthalenyl)-1-(aminomethyl)-4-benzamide;
N-(2-pyridinylmethyl)-N′-(6-methoxy-3,4-dihydronaphthalenyl)-1-(aminomethyl)-4-benzamide;
N-(2-pyridinylmethyl)-N′-(1H-imidazol-2-ylmethyl)-N′-(7-methoxy-1,2,3,4-tetrahydro-2-naphthalenyl)-1,4-benzenedimethanamine;
N-(2-pyridinylmethyl)-N′-(8-hydroxy-1,2,3,4-tetrahydro-2-naphthalenyl)-1,4-benzenedimethanamine;
N-(2-pyridinylmethyl)-N′-(1H-imidazol-2-ylmethyl)-N′-(8-hydroxy-1,2,3,4-tetrahydro-2-naphthalenyl)-1,4-benzenedimethanamine;
N-(2-pyridinylmethyl)-N′-(8-Fluoro-1,2,3,4-tetrahydro-2-naphthalenyl)-1,4-benzenedimethanamine;
N-(2-pyridinylmethyl)-N′-(1H-imidazol-2-ylmethyl)-N′-(8-Fluoro-1,2,3,4-tetrahydro-2-naphthalenyl)-1,4-benzenedimethanamine;
N-(2-pyridinylmethyl)-N′-(5,6,7,8-tetrahydro-7-quinolinyl)-1,4-benzenedimethanamine;
N-(2-pyridinylmethyl)-N′-(1H-imidazol-2-ylmethyl)-N′-(5,6,7,8-tetrahydro-7-quinolinyl)-1,4-benzenedimethanamine;
N-(2-pyridinylmethyl)-N′-[2-[(2-naphthalenylmethyl)amino]ethyl]-N′-(5,6,7,8-tetrahydro-8-quinolinyl)-1,4-benzenedimethanamine;
N-(2-pyridinylmethyl)-N′-[2-(isobutylamino)ethyl]-N′-(5,6,7,8-tetrahydro-8-quinolinyl)-1,4-benzenedimethanamine;
N-(2-pyridinylmethyl)-N′-[2-[(2-pyridinylmethyl)amino]ethyl]-N′-(5,6,7,8-tetrahydro-8-quinolinyl)-1,4-benzenedimethanamine;
N-(2-pyridinylmethyl)-N′-[2-[(2-furanylmethyl)amino]ethyl]-N′-(5,6,7,8-tetrahydro-8-quinolinyl)-1,4-benzenedimethanamine;
N-(2-pyridinylmethyl)-N′-(2-guanidinoethyl)-N′-(5,6,7,8-tetrahydro-8-quinolinyl)-1,4-benzenedimethanamine;
N-(2-pyridinylmethyl)-N′-[2-[bis-[(2-methoxy)phenylmethyl]amino]ethyl]-N′-(5,6,7,8-tetrahydro-8-quinolinyl)-1,4-benzenedimethanamine;
N-(2-pyridinylmethyl)-N′-[2-[(1H-imidazol-4-ylmethyl)amino]ethyl]-N′-(5,6,7,8-tetrahydro-8-quinolinyl)-1,4-benzenedimethanamine;
N-(2-pyridinylmethyl)-N′-[2-[(1H-imidazol-2-ylmethyl)amino]ethyl]-N′-(5,6,7,8-tetrahydro-8-quinolinyl)-1,4-benzenedimethanamine;
N-(2-pyridinylmethyl)-N′-[2-(phenylureido)ethyl]-N′-(5,6,7,8-tetrahydro-8-quinolinyl)-1,4-benzenedimethanamine;
N-(2-pyridinylmethyl)-N′-[[N″-(n-butyl)carboxamido]methyl]-N′-(5,6,7,8-tetrahydro-8-quinolinyl)-1,4-benzenedimethanamine;
N-(2-pyridinylmethyl)-N′-(carboxamidomethyl)-N′-(5,6,7,8-tetrahydro-8-quinolin yl)-1,4-benzenedimethanamine;
N-(2-pyridinylmethyl)-N′-[(N″-phenyl)carboxamidomethyl]-N′-(5,6,7,8-tetrahydro-8-quinolinyl)-1,4-benzenedimethanamine;
N-(2-pyridinylmethyl)-N′-(carboxymethyl)-N′-(5,6,7,8-tetrahydro-8-quinolinyl)-1,4-benzenedimethanamine;
N-(2-pyridinylmethyl)-N′-(phenylmethyl)-N′-(5,6,7,8-tetrahydro-8-quinolinyl)-1,4-benzenedimethanamine;
N-(2-pyridinylmethyl)-N′-(1H-benzimidazol-2-ylmethyl)-N′-(5,6,7,8-tetrahydro-8-quinolinyl)-1,4-benzenedimethanamine;
N-(2-pyridinylmethyl)-N′-(5,6-dimethyl-1H-benzimidazol-2-ylmethyl)-N′-(5,6,7,8-tetrahydro-8-quinolinyl)-1,4-benzenedimethanamine (hydrobromide salt);
N-(2-pyridinylmethyl)-N′-(5-nitro-1H-benzimidazol-2-ylmethyl)-N′-(5,6,7,8-tetrahydro-8-quinolinyl)-1,4-benzenedimethanamine;
N-(2-pyridinylmethyl)-N′-[(1H)-5-azabenzimidazol-2-ylmethyl]-N′-(5,6,7,8-tetrahydro-8-quinolinyl)-1,4-benzenedimethanamine;
N-(2-pyridinylmethyl)-N-(4-phenyl-1H-imidazol-2-ylmethyl)-N′-(5,6,7,8-tetrahydro-8-quinolinyl)-1,4-benzenedimethanamine;
N-(2-pyridinylmethyl)-N′-[2-(2-pyridinyl)ethyl]-N′-(5,6,7,8-tetrahydro-8-quinolin yl)-1,4-benzenedimethanamine;
N-(2-pyridinylmethyl)-N′-(2-benzoxazolyl)-N′-(5,6,7,8-tetrahydro-8-quinolinyl)-1,4-benzenedimethanamine;
N-(2-pyridinylmethyl)-N′-(trans-2-aminocyclohexyl)-N′-(5,6,7,8-tetrahydro-8-quinolinyl)-1,4-benzenedimethanamine;
N-(2-pyridinylmethyl)-N′-(2-phenylethyl)-N′-(5,6,7,8-tetrahydro-8-quinolinyl)-1,4-benzenedimethanamine;
N-(2-pyridinylmethyl)-N′-(3-phenylpropyl)-N′-(5,6,7,8-tetrahydro-8-quinolinyl)-1,4-benzenedimethanamine;
N-(2-pyridinylmethyl)-N′-(trans-2-aminocyclopentyl)-N′-(5,6,7,8-tetrahydro-8-quinolinyl)-1,4-benzenedimethanamine;
N-[[4-[[(2-pyridinylmethyl)amino]methyl]phenyl]methyl]-N-(5,6,7,8-tetrahydro-8-quinolinyl)-glycinamide;
N-[[4-[[(2-pyridinylmethyl)amino]methyl]phenyl]methyl]-N-(5,6,7,8-tetrahydro-8-quinolinyl)-(L)-alaninamide;
N-[[4-[[(2-pyridinylmethyl)amino]methyl]phenyl]methyl]-N-(5,6,7,8-tetrahydro-8-quinolinyl)-(L)-aspartamide;
N-[[4-[[(2-pyridinylmethyl)amino]methyl]phenyl]methyl]-N-(5,6,7,8-tetrahydro-8-quinolinyl)-pyrazinamide;
N-[[4-[[(2-pyridinylmethyl)amino]methyl]phenyl]methyl]-N-(5,6,7,8-tetrahydro-8-quinolinyl)-(L)-prolinamide;
N-[[4-[[(2-pyridinylmethyl)amino]methyl]phenyl]methyl]-N-(5,6,7,8-tetrahydro-8-quinolinyl)-(L)-lysinamide;
N-[[4-[[(2-pyridinylmethyl)amino]methyl]phenyl]methyl]-N-(5,6,7,8-tetrahydro-8-quinolinyl)-benzamide;
N-[[4-[[(2-pyridinylmethyl)amino]methyl]phenyl]methyl]-N-(5,6,7,8-tetrahydro-8-quinolinyl)-picolinamide;
N′-Benzyl-N-[[4-[[(2-pyridinylmethyl)amino]methyl]phenyl]methyl]-N-(5,6,7,8-tetrahydro-8-quinolinyl)-urea;
N′-phenyl-N-[[4-[[(2-pyridinylmethyl)amino]methyl]phenyl]methyl]-N-(5,6,7,8-tetrahydro-8-quinolinyl)-urea;
N-(6,7,8,9-tetrahydro-5H-cyclohepta[bacteriapyridin-9-yl)-4-[[(2-pyridinylmethyl)amino]methyl]benzamide;
N-(5,6,7,8-tetrahydro-8-quinolinyl)-4-[[(2-pyridinylmethyl)amino]methyl]benzamide;
N,N′-bis(2-pyridinylmethyl)-N′-(5,6,7,8-tetrahydro-8-quinolinyl)-1,4-benzenedimethanamine;
N,N′-bis(2-pyridinylmethyl)-N′-(6,7,8,9-tetrahydro-5H-cyclohepta[bacteriapyridin-9-yl)-1,4-benzenedimethanamine;
N,N′-bis(2-pyridinylmethyl)-N′-(6,7-dihydro-5H-cyclopenta[bacteriapyridin-7-yl)-1,4-benzenedimethanamine;
N,N′-bis(2-pyridinylmethyl)-N′-(1,2,3,4-tetrahydro-1-naphthalenyl)-1,4-benzenedimethanamine;
N,N′-bis(2-pyridinylmethyl)-N′-[(5,6,7,8-tetrahydro-8-quinolinyl)methyl]-1,4-benzenedimethanamine;
N,N′-bis(2-pyridinylmethyl)-N′[(6,7-dihydro-5H-cyclopenta[bacteriapyridin-7-yl)methyl]-1,4-benzenedimethanamine;
N-(2-pyridinylmethyl)-N-(2-methoxyethyl)-N′-(5,6,7,8-tetrahydro-8-quinolinyl)-1,4-benzenedimethanamine;
N-(2-pyridinylmethyl)-N-[2-(4-methoxyphenyl)ethyl]-N′-(5,6,7,8-tetrahydro-8-quinolinyl)-1,4-benzenedimethanamine;
N,N′-bis(2-pyridinylmethyl)-1,4-(5,6,7,8-tetrahydro-8-quinolinyl)benzenedimethanamine;
N-[(2,3-dimethoxyphenyl)methyl]-N′-(2-pyridinylmethyl)-N-(5,6,7,8-tetrahydro-8-quinolinyl)-1,4-benzenedimethanamine;
N,N′-bis(2-pyridinylmethyl)-N-[1-(N″-phenyl-N″-methylureido)-4-piperidinyl]-1,3-benzenedimethanamine;
N,N′-bis(2-pyridinylmethyl)-N—[N″-p-toluenesulfonylphenylalanyl)-4-piperidinyl]-1,3-benzenedimethanamine;
N,N′-bis(2-pyridinylmethyl)-N-[1-[3-(2-chlorophenyl)-5-methyl-isoxazol-4-oyl]-4-piperidinyl]-1,3-benzenedimethanamine;
N-[(2-hydroxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(5,6,7,8-tetrahydro-8-quinolinyl)-1,4-benzenedimethanamine;
N-[(4-acetamidophenyl)methyl]-N′-(2-pyridinylmethyl)-N-(5,6,7,8-tetrahydro-8-quinolinyl)-1,4-benzenedimethanamine;
N-[(4-phenoxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(1-methyl-2-carboxamido)ethyl]-N,N′-bis(2-pyridinylmethyl)-1,3-benzenedimethanamine;
N-[(4-benzyloxyphenyl)methyl]-N′-(2-pyridinylmethyl)-N-(6,7,8,9-tetrahydro-5H-cyclohepta[bacteriapyridin-9-yl)-1,4-benzenedimethanamine;

N-[(thiophene-2-yl)methyl]-N′-(2-pyridinylmethyl)-N-(6,7,8,9-tetrahydro-5H-cyclohepta[bacteriapyridin-9-yl)-1,4-benzenedimethanamine;
N-[1-(benzyl)-3-pyrrolidinyl]-N,N′-bis(2-pyridinylmethyl)-1,3-benzenedimethanamine;
N-[[1-methyl-3-(pyrazol-3-yl)]propyl]-N,N′-bis(2-pyridinylmethyl)-1,3-benzenedimethanamine;
N-[1-(phenyl)ethyl]-N,N′-bis(2-pyridinylmethyl)-1,3-benzenedimethanamine;
N-[(3,4-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[1-benzyl-3-carboxymethyl-4-piperidinyl]-N,N′-bis(2-pyridinylmethyl)-1,3-benzenedimethanamine;
N-[(3,4-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(6,7,8,9-tetrahydro-5H-cyclohepta[b]pyridin-9-yl)-1,4-benzenedimethanamine;
N-[[1-methyl-2-(2-tolyl)carboxamido]ethyl]-N,N′-bis(2-pyridinylmethyl)-1,3-benzenedimethanamine;
N-[(1,5-dimethyl-2-phenyl-3-pyrazolinone-4-yl)methyl]-N′-(2-pyridinylmethyl)-N-(5,6,7,8-tetrahydro-8-quinolinyl)-1,4-benzenedimethanamine;
N-[(4-propoxyphenyl)methyl]-N′-(2-pyridinylmethyl)-N-(6,7,8,9-tetrahydro-5H-cyclohepta[b]pyridin-9-yl)-1,4-benzenedimethanamine;
N-(1-phenyl-3,5-dimethylpyrazolin-4-ylmethyl)-N′-(2-pyridinylmethyl)-N-(5,6,7,8-tetrahydro-8-quinolinyl)-1,4-benzenedimethanamine;
N-[1H-imidazol-4-ylmethyl]-N,N′-bis(2-pyridinylmethyl)-1,3-benzenedimethanamine;
N-[(3-methoxy-4,5-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(5,6,7,8-tetrahydro-8-quinolinyl)-1,4-benzenedimethanamine;
N-(5-ethylthiophene-2-ylmethyl)-N′-(2-pyridinylmethyl)-N-(6,7,8,9-tetrahydro-5H-cyclohepta[b]pyridin-9-yl)-1,4-benzenedimethanamine;
N-(5-ethylthiophene-2-ylmethyl)-N′-(2-pyridinylmethyl)-N-(5,6,7,8-tetrahydro-8-quinolinyl)-1,4-benzenedimethanamine;
N-[(2,6-difluorophenyl)methyl]-N′-(2-pyridinylmethyl)-N-(6,7,8,9-tetrahydro-5H-cyclohepta[b]pyridin-9-yl)-1,4-benzenedimethanamine;
N-[(2,6-difluorophenyl)methyl]-N′-(2-pyridinylmethyl)-N-(5,6,7,8-tetrahydro-8-quinolinyl)-1,4-benzenedimethanamine;
N-[(2-difluoromethoxyphenyl)methyl]-N′-(2-pyridinylmethyl)-N-(6,7,8,9-tetrahydro-5H-cyclohepta[b]pyridin-9-yl)-1,4-benzenedimethanamine;
N-(2-difluoromethoxyphenylmethyl)-N′-(2-pyridinylmethyl)-N-(5,6,7,8-tetrahydro-8-quinolinyl)-1,4-benzenedimethanamine;
N-(1,4-benzodioxan-6-ylmethyl)-N′-(2-pyridinylmethyl)-N-(6,7,8,9-tetrahydro-5H-cyclohepta[b]pyridin-9-yl)-1,4-benzenedimethanamine;
N,N′-bis(2-pyridinylmethyl)-N-[1-(N″-phenyl-N″-methylureido)-4-piperidinyl]-1,4-benzenedimethanamine;
N,N′-bis(2-pyridinylmethyl)-N—[N″-p-toluenesulfonylphenylalanyl)-4-piperidinyl]-1,4-benzenedimethanamine;
N-[1-(3-pyridinecarboxamido)-4-piperidinyl]-N,N′-bis(2-pyridinylmethyl)-1,4-benzenedimethanamine;
N-[1-(cyclopropylcarboxamido)-4-piperidinyl]-N,N′-bis(2-pyridinylmethyl)-1,4-benzenedimethanamine;
N-[1-(1-phenylcyclopropylcarboxamido)-4-piperidinyl]-N,N′-bis(2-pyridinylmethyl)-1,4-benzenedimethanamine;
N-(1,4-benzodioxan-6-ylmethyl)-N′-(2-pyridinylmethyl)-N-(5,6,7,8-tetrahydro-8-quinolinyl)-1,4-benzenedimethanamine;
N-[1-[3-(2-chlorophenyl)-5-methyl-isoxazol-4-carboxamido]-4-piperidinyl]-N,N′-bis(2-pyridinylmethyl)-1,4-benzenedimethanamine;
N-[1-(2-thiomethylpyridine-3-carboxamido)-4-piperidinyl]-N,N′-bis(2-pyridinylmethyl)-1,4-benzenedimethanamine;
N-[(2,4-difluorophenyl)methyl]-N′-(2-pyridinylmethyl)-N-(5,6,7,8-tetrahydro-8-quinolinyl)-1,4-benzenedimethanamine;
N-(1-methylpyrrol-2-ylmethyl)-N′-(2-pyridinylmethyl)-N-(5,6,7,8-tetrahydro-8-quinolinyl)-1,4-benzenedimethanamine;
N-[(2-hydroxyphenyl)methyl]-N′-(2-pyridinylmethyl)-N-(5,6,7,8-tetrahydro-8-quinolinyl)-1,4-benzenedimethanamine;
N-[(3-methoxy-4,5-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(5,6,7,8-tetrahydro-8-quinolinyl)-1,4-benzenedimethanamine;
N-[2-(N″-morpholinomethyl)-1-cyclopentyl]-N,N′-bis(2-pyridinylmethyl)-1,4-benzenedimethanamine;
N-[(1-methyl-3-piperidinyl)propyl]-N,N′-bis(2-pyridinylmethyl)-1,4-benzenedimethanamine;
N-(1-methylbenzimidazol-2-ylmethyl)-N′-(2-pyridinylmethyl)-N-(5,6,7,8-tetrahydro-8-quinolinyl)-1,4-benzenedimethanamine;
N-[1-(benzyl)-3-pyrrolidinyl]-N,N′-bis(2-pyridinylmethyl)-1,4-benzenedimethanamine;

N-[[(1-phenyl-3-(N″-morpholino)]propyl]-N,N′-bis(2-pyridinylmethyl)-1,4-benzenedimethanamine;
N-[1-(iso-propyl)-4-piperidinyl]-N,N′-bis(2-pyridinylmethyl)-1,4-benzenedimethanamine;
N-[1-(ethoxycarbonyl)-4-piperidinyl]-N′-(2-pyridinylmethyl)-N-(5,6,7,8-tetrahydro-8-quinolinyl)-1,4-benzenedimethanamine;
N-[(1-methyl-3-pyrazolyl)propyl]-N′-(2-pyridinylmethyl)-N-(5,6,7,8-tetrahydro-8-quinolinyl)-1,4-benzenedimethanamine;
N-[1-methyl-2-(N″,N″-diethylcarboxamido)ethyl]-N,N′-bis(2-pyridinylmethyl)-1,4-benzenedimethanamine;
N-[(1-methyl-2-phenylsulfonyl)ethyl]-N′-(2-pyridinylmethyl)-N-(5,6,7,8-tetrahydro-8-quinolinyl)-1,4-benzenedimethanamine;
N-[(2-chloro-4,5-methylenedioxyphenyl)methyl]-N′-(2-pyridinylmethyl)-N-(5,6,7,8-tetrahydro-8-quinolinyl)-1,4-benzenedimethanamine;
N-[1-methyl-2-[N″-(4-chlorophenyl)carboxamido]ethyl]-N′-(2-pyridinylmethyl)-N-(5,6,7,8-tetrahydro-8-quinolinyl)-1,4-benzenedimethanamine;
N-(1-acetoxyindol-3-ylmethyl)-N′-(2-pyridinylmethyl)-N-(6,7,8,9-tetrahydro-5H-cyclohepta[b]pyridin-9-yl)-1,4-benzenedimethanamine;
N-[(3-benzyloxy-4-methoxyphenyl)methyl]-N′-(2-pyridinylmethyl)-N-(6,7,8,9-tetrahydro-5H-cyclohepta[b]pyridin-9-yl)-1,4-benzenedimethanamine;
N-(3-quinolylmethyl)-N′-(2-pyridinylmethyl)-N-(5,6,7,8-tetrahydro-8-quinolinyl)-1,4-benzenedimethanamine;
N-[(8-hydroxy)-2-quinolylmethyl]-N′-(2-pyridinylmethyl)-N-(6,7,8,9-tetrahydro-5H-cyclohepta[b]pyridin-9-yl)-1,4-benzenedimethanamine;
N-(2-quinolylmethyl)-N′-(2-pyridinylmethyl)-N-(6,7,8,9-tetrahydro-5H-cyclohepta[b]pyridin-9-yl)-1,4-benzenedimethanamine;
N-[(4-acetamidophenyl)methyl]-N′-(2-pyridinylmethyl)-N-(6,7,8,9-tetrahydro-5H-cyclohepta[b]pyridin-9-yl)-1,4-benzenedimethanamine;
N-[1H-imidazol-2-ylmethyl]-N,N′-bis(2-pyridinylmethyl)-1,4-benzenedimethanamine;
N-(3-quinolylmethyl)-N′-(2-pyridinylmethyl)-N-(6,7,8,9-tetrahydro-5H-cyclohepta[b]pyridin-9-yl)-1,4-benzenedimethanamine;
N-(2-thiazolylmethyl)-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(6,7,8,9-tetrahydro-5H-cyclohepta[b]pyridin-9-yl)-1,4-benzenedimethanamine;
N-[(5-benzyloxy)benzo[b]pyrrol-3-ylmethyl]-N,N′-bis(2-pyridinylmethyl)-1,4-benzenedimethanamine;
N-(1-methylpyrazol-2-ylmethyl)-N′-(2-pyridinylmethyl)-N-(6,7,8,9-tetrahydro-5H-cyclohepta[b]pyridin-9-yl)-1,4-benzenedimethanamine;
N-[(4-methyl)-1H-imidazol-5-ylmethyl]-N,N′-bis(2-pyridinylmethyl)-1,4-benzenedimethanamine;
N-[[(4-dimethylamino)-1-napthalenyl]methyl]-N,N′-bis(2-pyridinylmethyl)-1,4-benzenedimethanamine;
N-[1,5-dimethyl-2-phenyl-3-pyrazolinone-4-ylmethyl]-N,N′-bis(2-pyridinylmethyl)-1,4-benzenedimethanamine;
N-[1-[(1-acetyl-2-(R)-prolinyl]-4-piperidinyl]-N-[2-(2-pyridinyl)ethyl]-N′-(2-pyridinylmethyl)-1,3-benzenedimethanamine;
N-[1-[2-acetamidobenzoyl-4-piperidinyl]-4-piperidinyl]-N-[2-(2-pyridinyl)ethyl]-N′-(2-pyridinylmethyl)-1,3-benzenedimethanamine;
N-[(2-cyano-2-phenyl)ethyl]-N′-(2-pyridinylmethyl)-N-(6,7,8,9-tetrahydro-5H-cyclohepta[b]pyridin-9-yl)-1,4-benzenedimethanamine;

N—[(N″-acetyltryptophanyl)-4-piperidinyl]-N-[2-(2-pyridinyl)ethyl]-N′-(2-pyridinylmethyl)-1,3-benzenedimethanamine;

N—[(N″-benzoylvalinyl)-4-piperidinyl]-N-[2-(2-pyridinyl)ethyl]-N′-(2-pyridinylmethyl)-1,3-benzenedimethanamine;
N-[(4-dimethylaminophenyl)methyl]-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(5,6,7,8-tetrahydro-8-quinolinyl)-1,4-benzenedimethanamine;
N-(1-methylbenzimadazol-2-ylmethyl)-N′-(2-pyridinylmethyl)-N-(6,7,8,9-tetrahydro-5H-cyclohepta[b]pyridin-9-yl)-1,4-benzenedimethanamine;
N-[1-butyl-4-piperidinyl]-N-[2-(2-pyridinyl)ethyl]-N′-(2-pyridinylmethyl)-1,3-benzenedimethanamine;
N-[1-benzoyl-4-piperidinyl]-N-[2-(2-pyridinyl)ethyl]-N′-(2-pyridinylmethyl)-1,3-benzenedimethanamine;
N-[1-(benzyl)-3-pyrrolidinyl]-N-[2-(2-pyridinyl)ethyl]-N′-(2-pyridinylmethyl)-1,3-benzenedimethanamine;
N-[(1-methyl)benzo[b]pyrrol-3-ylmethyl]-N-[2-(2-pyridinyl)ethyl]-N′-(2-pyridinylmethyl)-1,3-benzenedimethanamine;
N-[1H-imidazol-4-ylmethyl]-N-[2-(2-pyridinyl)ethyl]-N′-(2-pyridinylmethyl)-1,3-benzenedimethanamine;
N-[1-(benzyl)-4-piperidinyl]-N-[2-(2-pyridinyl)ethyl]-N′-(2-pyridinylmethyl)-1,4-benzenedimethanamine;
N-[1-methylbenzimidazol-2-ylmethyl]-N-[2-(2-pyridinyl)ethyl]-N′-(2-pyridinylmethyl)-1,4-benzenedimethanamine;
N-[(2-phenyl)benzo[b]pyrrol-3-ylmethyl]-N-[2-(2-pyridinyl)ethyl]-N′-(2-pyridinylmethyl)-1,4-benzenedimethanamine;
N-[(6-methylpyridin-2-yl)methyl]-N′-(2-pyridinylmethyl)-N-(5,6,7,8-tetrahydro-8-quinolinyl)-1,4-benzenedimethanamine;
N-(3-methyl-1H-pyrazol-5-ylmethyl)-N′-(2-pyridinylmethyl)-N-(5,6,7,8-tetrahydro-8-quinolinyl)-1,3-benzenedimethanamine;
N-[(2-methoxyphenyl)methyl]-N′-(2-pyridinylmethyl)-N-(5,6,7,8-tetrahydro-8-quinolinyl)-1,3-benzenedimethanamine;
N-[(2-ethoxyphenyl)methyl]-N′-(2-pyridinylmethyl)-N-(6,7,8,9-tetrahydro-5H-cyclohepta[b]pyridin-9-yl)-1,3-benzenedimethanamine;
N-(benzyloxyethyl)-N′-(2-pyridinylmethyl)-N-(5,6,7,8-tetrahydro-8-quinolinyl)-1,3-benzenedimethanamine;
N-[(2-ethoxy-1-naphthalenyl)methyl]-N′-(2-pyridinylmethyl)-N-(5,6,7,8-tetrahydro-8-quinolinyl)-1,3-benzenedimethanamine;
N-[(6-methylpyridin-2-yl)methyl]-N′-(2-pyridinylmethyl)-N-(5,6,7,8-tetrahydro-8-quinolinyl)-1,3-benzenedimethanamine;
1-[[4-[[(2-pyridinylmethyl)amino]methyl]phenyl]methyl]guanidine;
N-(2-pyridinylmethyl)-N-(8-methyl-8-azabicyclo[3.2.1]octan-3-yl)-1,4-benzenedimethanamine;
1-[[4-[[(2-pyridinylmethyl)amino]methyl]phenyl]methyl]homopiperazine;
1-[[3-[[(2-pyridinylmethyl)amino]methyl]phenyl]methyl]homopiperazine;

trans and cis-1-[[4-[[(2-pyridinylmethyl)amino]methyl]phenyl]methyl]-3,5-piperidinediamine;
N,N′-[1,4-Phenylenebis(methylene)]bis-4-(2-pyrimidyl)piperazine;
1-[[4-[[(2-pyridinylmethyl)amino]methyl]phenyl]methyl]-1-(2-pyridinyl)methylamine;
2-(2-pyridinyl)-5-[[(2-pyridinylmethyl)amino]methyl]-1,2,3,4-tetrahydroisoquinoline;
1-[[4-[[(2-pyridinylmethyl)amino]methyl]phenyl]methyl]-3,4-diaminopyrrolidine;
1-[[4-[[(2-pyridinylmethyl)amino]methyl]phenyl]methyl]-3,4-diacetylaminopyrrolidine;
8-[[4-[[(2-pyridinylmethyl)amino]methyl]phenyl]methyl]-2,5,8-triaza-3-oxabicyclo[4.3.0]nonane; and
8-[[4-[[(2-pyridinylmethyl)amino]methyl]phenyl]methyl]-2,5,8-triazabicyclo[4.3.0]nonane.

Compounds having formula (2A) and (2B) and methods for synthesizing such compounds are set forth in WO 00/56729, incorporated herein by reference.

Other CXCR4 antagonists are compounds of formula (3):

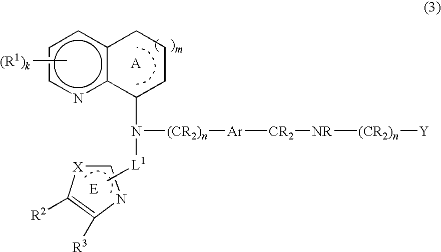

or the salts, prodrugs and stereochemical forms thereof, wherein:

Ring A optionally comprises a heteroatom selected from N, O and S;

the dotted lines represent optional unsaturation;

R¹ is halo, nitro, cyano, optionally substituted hydroxy, optionally substituted thiol, optionally substituted amino, carboxylate, carboxamide, sulfonate, sulfonamide, C2-4 alkanoyl, alkylsulfonyl, or aroyl;

R² and R³ are independently H, an optionally halogenated C₁₋₄ alkyl, an optionally substituted aryl or heterocyclic group, or R² and R³ together with ring E may form a substituted or unsubstituted 5-7 membered ring;

k is 0-4;

m is 0-2;

L¹ is a covalent bond of C₁₋₆ alkyl optionally containing N or O;

X is unsubstituted or substituted C, N; or O or S;

Ar is phenylene;

each n is independently 0-2;

each R is independently H or alkyl (1-6C); and

Y is a fused or unfused aromatic or heteroaromatic ring, or a 5-6 membered heterocyclic group.

In the above formula (3), Y may be a substituted or unsubstituted benzene, napthalene, dihydronapthalene, tetrahydronapthalene, pyridine, quinoline, isoquinoline, imidazole, benzimidazole, azabenzimidazole, benzotriazole, furan, benzofuran, thiazole, benzothiazole, oxazole, benzoxazole, pyrrole, indole, indoline, indazole, pyrrolidine, pyrrolidone, pyrroline, piperidine, piperazine, tetrahydroquinoline, tetrahydroisoquinoline, pyrazole, thiophene, isoxazole, isothiazole, triazole, tetrazole, oxadiazole, thiadiazole, morpholine, thiamorpholine, pyrazolidine, imidazolidine, imidazoline, tetrahydropyran, dihydropyran, benzopyran, dioxane, dithiane, tetrahydrofuran, tetrahydrothiophene, dihydrofuran, or dihydrothiophene.

In the above formula (3), L¹ may be linked to position 2 of ring E. The dotted line in ring E may further represent a double bond between the nitrogen shown and position 2. In one example, R² and R³ are connected so as to form a benzosubstituent to ring E.

In the above formula (3), ring A may be saturated. In some examples, m is 1 and k is 0 or 1.

The CXCR4 antagonists may also have formula (3A):

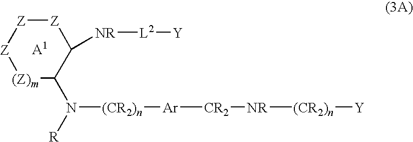

or the salts, prodrugs and stereochemical forms thereof, wherein:

R, m, n, Ar, and each Y are defined as in formula (3);

L² is a covalent bond or C₁₋₆ alkyl optionally containing N or O;

and each Z is independently CR₂, NR, O or S, with the proviso that only two Z can be other than CR₂.

In the above formula (3A), L² may be methylene or ethylene. In one example, m is 1 and all Z embodiments are CR₂, particularly CH₂.

In the above formula (3A), each Y may be pyrimidyl, pyridyl, phenyl, benzimidazole or benzoxazole.

Other CXCR4 antagonists have formula (3B):

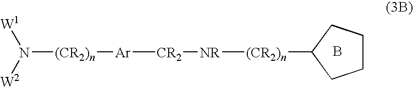

or the salts, prodrugs and stereochemical forms thereof, wherein:

W¹ is a monocyclic (5-6 membered) or fused bicyclic (8-12 membered) unsubstituted or substituted ring system containing at least one heteroatom selected from N, O and S;

W² is H, or is selected from the group consisting of: an optionally substituted C₁₋₆ alkyl group; a C₀₋₆ alkyl group substituted with an optionally substituted aromatic or heterocyclic group; an optionally substituted C₀₋₆ alkylamino or C₃₋₇ cycloalkylamino group; and an optionally substituted carbonyl group or sulfonyl;

Ar, R and n are defined as in Formula (3), and

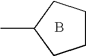

is a saturated or unsaturated 5-membered ring containing 1-2 heteroatoms selected from N, O and S.

Other CXCR4 antagonists have formula (3C):

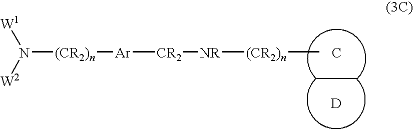

or the salts, prodrugs or stereochemical forms thereof, wherein:

W¹ is phenyl, pyridyl, pyridimyl, imidazolyl, thiophenylyl, and a fused ring system optionally having a heteroatom selected from N, O and S;

W² is H;

Ar, R and n are defined as in formula (3); and

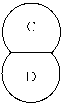

represents a fused ring system of 10 members, optionally containing 1 or 2 heteroatoms selected from N, O and S.

Compounds having formula (3), and (3A)-(3C) and methods for synthesizing such compounds are set forth in WO 02/22600, which is incorporated herein by reference.

Other CXCR4 antagonists have formula (4):

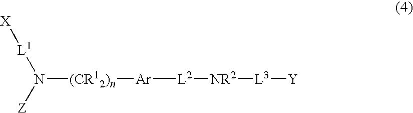

or the salts, prodrugs and stereochemical forms thereof, wherein:

X is a monocyclic (5-6 membered) or fused bicyclic (9-12 membered) unsubstituted or substituted ring system containing at least one heteroatom selected from N, O and S;

Z is H, or is an optionally substituted 5-6 membered monocyclic or 9-12 membered fused bicyclic ring system containing N, O or S;

Ar is an optionally substituted aromatic or heteroaromatic ring;

each of L¹, L² and L³ is independently a bond, CO, SO₂, or CH₂, wherein at least one of L and L¹ must comprise CO or SO₂; and wherein L¹ can also be alkylene (2-5C) wherein one or two C may optionally be replaced by N and which alkylene may itself optionally be substituted by a bridge alkylene (3-4C); L² and L³ also may be, independently, SO₂NH, CONH, SO₂NHCH₂ or CONHCH₂;

n is 0, 1 or 2;

each R¹ and R² is independently H or straight or branched chain or cyclic alkyl (1-6C) which may optionally be substituted, and wherein R² may be alkylene coupled to Y; and

Y comprises at least one aromatic or heteroaromatic or other heterocyclic substituted or unsubstituted ring coupled directly to L³.

In the above formula (4), X may be dihydroquinoline, tetrahydroquinoline, pyranopyridine, dihydropyranopyridine, thiapyranopyridine, dihydrothiapyranopyridine, dihydronaphthyridine, tetrahydronaphthyridine, imidazolyl, oxazolyl, thiazolyl, benzimidazolyl, benzothiazolyl, or benzoxazolyl.

In the above formula (4), L¹ may be alkylene (2-5C) wherein one C may optionally be replaced by N and which may optionally be substituted by a bridging alkylene (3-4C). For example, L¹ may be alkylene, CO or SO₂, and X is an optionally substituted imidazole, oxazole, thiazole, benzimidazole, benzothiazole, or benzoxazole. Alternatively, L¹ may be a bond, and X is substituted or unsubstituted dihydroquinoline, tetrahydroquinoline, pyranopyridine, dihydropyranopyridine, thiapyranopyridine, dihydrothiapyranopyridine, dihydronaphthyridine, or tetrahydronaphthyridine.

In the above formula (4), Z may be hydrogen.

In the above formula (4), Y may be an optionally substituted imidazole, benzimidazole, pyridine, pyridine, pyrimidine, or phenyl, wherein the ring nitrogen may optionally be oxidized. For example, Y may be substituted with halogen, nitrile, alkyl, —OR, —SR, —NR₂, —NRCOR, —OOCR, —COR, —CONR₂, —COOR, —NO₂, —NOH, —CF₃, where R is H or alkyl (1-6C).

In the above formula (4), each X or Z may optionally be substituted by halo, nitro, cyano, carboxy, C1-10 alkyl, C2-10 alkenyl, C3-10 cycloalkyl, hydroxy, thiol, amino, acyl, carboxylate, carbamate, carboxamide, sulfonamide, a carbonyl or sulfonyl binding to a hydrogen, or substituted with a C1-10-alkyl, C2-10 alkenyl, C3-7 cycloalkyl or a 5-6 membered monocyclic aromatic group; or X or Z may optionally be substituted by a 5-6 membered monocyclic aromatic group, naphthyl or a 5-6 membered heterocyclic ring;

Other CXCR4 antagonists have formula (4A):

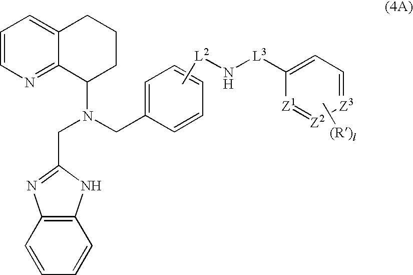

or formula (4B):

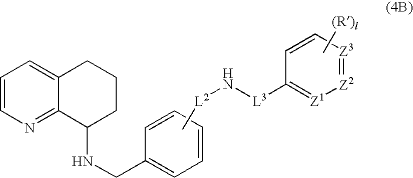

wherein 1 is 0-3, and R′ is OH, MeO, SH SMe, CN, CO₂Me, F, Cl, Br, NO₂, CH₃CO, NH₂, NHCH₃, N(CH₃)₂, CH₃CONH, CH₃SO₂NH, CONH₂, SO₂NH₂, CF₃, or Me;

each of Z¹, Z² and Z³ is independently CH, CR′ or N, wherein only two of said Z¹, Z² and Z³ can be N;

and L² and L³ are as defined in formula (4).

In the above formula (4A) or (4B), all of Z¹, Z² and Z³ may be CH or CR′. In one example, Z is N and L³ is CO. Furthermore, one of L² and L³ may be SO₂ and the other is a bond or CH₂. Alternatively, one of L and L³ is CO and the other is a bond or CH₂.

In another embodiment, the compound for use in the methods of the present invention has formula (4C):

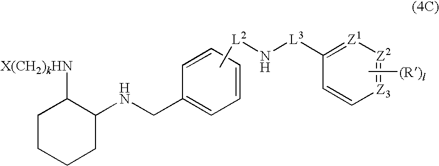

wherein 1 is 0-3, and R′ is OH, MeO, SH SMe, CN, CO₂Me, F, Cl, Br, NO₂, CH₃CO, NH₂, NHCH₃, N(CH₃)₂, CH₃CONH, CH₃SO₂NH, CONH₂, SO₂NH₂, CF₃, or Me;

k is 0-2;

each of Z¹, Z² and Z³ is independently CH, CR′ or N, wherein only two of said Z¹, Z² and Z³ can be N;

and X, L² and L³ are as defined in formula (4).

In the above formula (4C), all of Z¹, Z² and Z³ may be CH or CR′. In one example, Z is N and L³ is CO. Furthermore, one of L² and L³ may be SO₂ and the other is a bond or CH₂. Alternatively, one of L² and L³ may be CO and the other is a bond or CH₂.

Compounds having formula (4), and (4A)-(4C) and methods of synthesizing such compounds are set forth in WO 02/22599, which is incorporated herein by reference.

Other CXCR4 antagonists have formula (5):

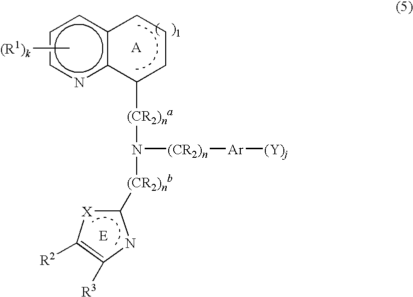

or the salts, prodrugs and stereoisomeric forms thereof;

Ring A optionally comprises a heteroatom selected from N, O and S;

the dotted lines represent optional unsaturation;

R¹, R² and R³ are independently H, halo, substituted or unsubstituted alkyl, hydroxyl, amino, thiol, or acyl; or R² and R³ may together form a benzo ring;

k is 0-4;

l is 0, 1, or 2;

X is unsubstituted or substituted C or N; or is O or S;

Ar is the residue of an aromatic or heteroaromatic moiety;

each n is independently 0-2;

each R is independently H or alkyl (1-6C);

j is 0-3; and

each Y is independently selected from the group consisting of halo, OR; SH; SO;

SO₂;

optionally substituted phenyl;

—(CR₂)ₘOR;

—(CR₂)ₘCOR;

—(CR₂)ₘCOOR;

—(CR₂)ₘN═CH—NR₂;

—(CR₂)ₘCONHNHR;

—(CR₂)ₘCN;

—(CR₂)ₘNR⁵₂;

—(CR₂)ₘNR(CR₂)ₘNRR⁴;

(CR₂)ₘNR(CR₂)ₘNR(CR₂)ₘNR⁵₂;

—(CR₂)ₘCO(CR₂)ₘNR⁵₂;

(CR₂)ₘCO(CR₂)ₘNR(CR₂)ₘNRR⁴;

—(CR₂)ₘCO(CR₂)ₘNR(CR₂)ₘNR(CR₂)ₘNR⁵₂;

—(CR₂)ₘNRCO(CR₂)ₘNRR₄;

—(CR₂)ₘNRCO(CR₂)ₘNR(CR₂)ₘNR⁵₂;

—(CR₂)ₘNRCO(CR₂)ₘNR(CR₂)ₘNR(CR₂)ₘNR(CR₂)ₘNR⁵₂;

—(CR₂)ₘNROH;

—(CR₂)ₘCONROH;

—(CR₂)ₘCR═NOH;

—NHNHR;

—CH═N-Z; and

guanidino or amidino, each of which may be linked to Y through a (CR₂)ₘ moiety;

wherein R is H or alkyl (1-6C), each m is independently 0-4, and each R⁴ and each R⁵ is independently H, alkyl (1-6C), alkenyl (2-6C), alkynyl (2-6C), or acyl (1-6C), each optionally substituted by one or more nonaromatic, nonheterocyclic substituent(s), wherein two R⁵ may be connected to form a cyclic amine optionally containing one or more additional heteroatoms selected from N, O and S;

a indicates the linker between Ring A and N;

b indicates the linker between ring E and the N; and

wherein Z is an aromatic or heteroaromatic moiety containing 5-12 ring members.

In the above formula (5), Ar may be a 5-6 membered monocyclic ring or a 9-12 membered fused ring system. For example, Ar may be benzene, naphthalene, dihydronaphthalene, tetrahydronaphthalene, pyridine, pyrimidine, quinoline, isoquinoline, imidazole, benzimidazole, azabenzimidazole, benzotriazole, furan, benzofuran, thiazole, benzothiazole, oxazole, benzoxazole, pyrrole, indole, imidazole, tetrahydroquinoline, tetrahydroisoquinoline, pyrazole, thiophene, isoxazole, isothiazole, triazole, tetrazole, oxadiazole, thiadiazole, imidazoline, and benzopyran. In particular examples, Ar is benzene, benzimidazole, benzothiazole, imidazole, oxazole, benztriazole, thiazole, pyridine, or pyrimidine. In one embodiment, at least one Y is —(CR₂)ₘNR⁵₂.

In the above formula (5), R² and R³ taken together may form a benzo substituent. In one embodiment, X is N and ring E comprises a pi bond coupled to one N. In one embodiment, ring E is coupled to the remainder of the molecule at position 2.

In the above formula (5), ring A may be saturated and l is 1. In one example, k is 0-1. In other examples, the ring system which includes A is tetrahydroquinoline or a substituted form thereof.

In the above formula (5), one of (CR₂)^{a}ₙ and (CR₂)^{b}ₙ may be CH₂ and the other is a bond. For example, (CR₂)^{a}ₙ may be a bond and (CR₂)^{b}ₙ is CH₂.

Compounds having formula (5) and methods for synthesizing such compounds are set forth in WO 02/34745, which is incorporated herein by reference.

Other CXCR4 antagonists have formula (6):

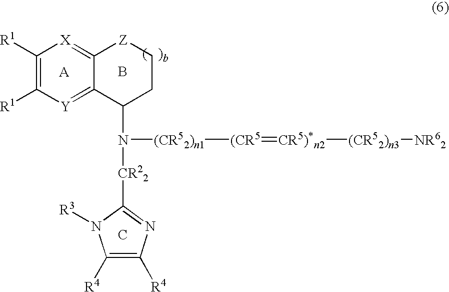

or the salts, prodrugs and stereoisomeric forms thereof,

wherein X and Y are independently N or CR¹;

Z is S, O, NR¹ or CR¹₂;

each R¹-R⁶ is independently H, halo, O(C═O)R, NR(C═O)R, OR, SR, NR₂, COOR, CONR₂, where R is H or optionally substituted alkyl, alkenyl, alkynyl or aryl; or

each R¹-R⁶ is alkyl (C₁₋₁₀), alkenyl (C₂₋₁₀), alkynyl (C₂₋₁₀), aryl (C₅₋₁₂), arylalkyl, arylalkenyl, or arylalkynyl, each optionally containing substituted and optionally containing O, S, or N; or an optionally substituted acyl, arylacyl, alkyl-alkenyl-, alkynyl- or arylsulfonyl wherein each alkyl, alkenyl, alkynyl or aryl moiety may contain O, O or N;

n1 is 0-4;

n2 is 0-1, wherein the * signifies C≡C may be substituted for CR⁵═CR⁵;

n3 is 0-4;

wherein n1+n2+n3 is greater than or equal to 2;

b is 0-2;

wherein the following combinations of R groups may be coupled to generate a ring, which ring may be saturated or unsaturated:

R²+R²

one R²+R³

R³+one R⁴,

R⁴+R⁴,

one R+another R⁵,

one R+one R⁶, and

R⁶+R⁶;

wherein the ring may not be aromatic when the participants in ring formation are two R⁵; and

wherein when n2 is 1, neither n1 nor n3 can be 0.

Other CXCR4 antagonists have formula (6A):

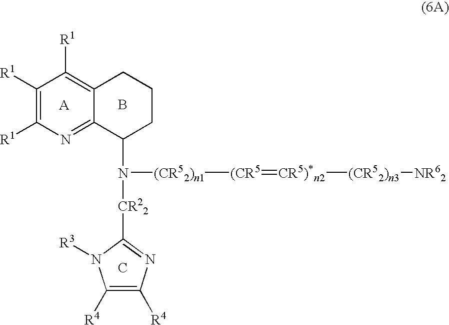

or the salts, prodrugs and stereoisomeric forms thereof,

wherein R¹-R⁶ and n1-n3 are as defined in formula (6).

Other antagonists have formula (6B) or formula (6C):

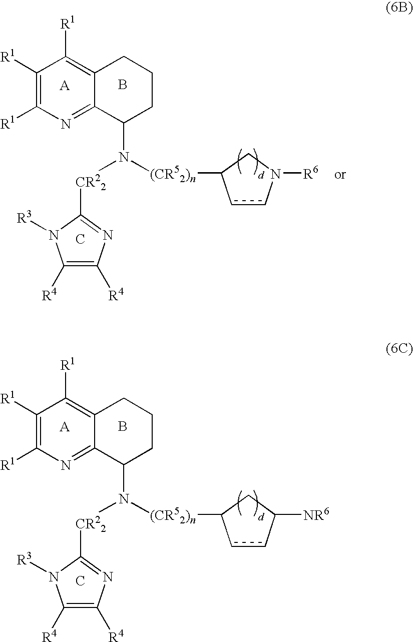

or the salts, prodrugs and stereoisomeric forms thereof,

wherein n is 0-1;

d is 0-3; the dotted line is an optional π bond; and

R¹-R⁶ are defined as in formula (6).

In yet another embodiment, the compounds for use in the methods of the present invention have formula (6D):

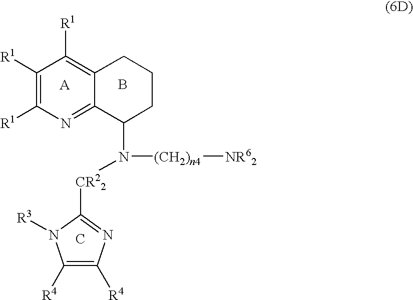

or the salts, prodrugs and stereoisomeric forms thereof,

wherein R¹-R⁶ are defined as in formula (6), and n4 is 2-6.

In the above formula (6) or (6A)-(6D), each R¹ may be H, halo, alkyl, alkoxy, or CF₃. In one embodiment, each R² is H or alkyl. In another embodiment, each R³ is H, alkyl, alkenyl, arylalkyl, or aryl.

In the above formula (6) or (6A)-(6D), each R⁴ may be H, alkyl or aryl.

Alternatively, two R⁴ may form an optionally substituted aromatic or heteroaromatic ring. For example, two R⁴ may form a phenyl or pyridyl ring, which may be substituted with halo, alkyl, halogenated alkyl, hydroxy, or alkoxy.

In the above formula (6) or (6A)-(6D), each R⁵ may be H, alkyl, or alkenyl, wherein said alkyl or alkenyl may optionally be substituted. In one embodiment, the alkyl or alkenyl substituents on a single carbon, or on nonadjacent or adjacent carbons, form a saturated or unsaturated ring. In one example, the substituents form a nonaromatic ring.

In another embodiment, one R⁵ is an oxime, an alkylated oxime, alkylated hydroxylamine, hydroxylamine or halo.

In the above formula (6) or (6A)-(6D), each R⁶ may independently H, or an arylalkyl or arylsulfonyl, wherein the aryl moiety may comprise a heteroatom; or two R⁶ may comprise a guanidyl, carbonyl, or carbamino group. In one embodiment, two R⁶ together, or one R⁵ and one R⁶ together may form a saturated, unsaturated or aromatic ring, wherein each ring may optionally contain N, S or O.

Compounds having formula (6) and methods for synthesizing such compounds are set forth in WO 03/055876, which is incorporated herein by reference.

The CXCR4 antagonist may have formula (7):

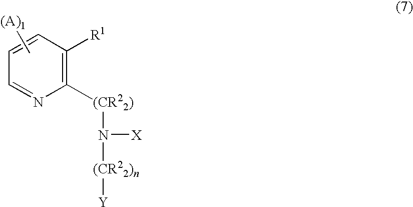

or the salts, prodrugs and stereoisomeric forms thereof,

wherein X is (CR³₂)ₒ—(CR³═CR³)ₚ—(CR³₂)_{q}—NR⁵₂; (CR³₂)ᵣ—R⁴; or an optionally substituted benzyl, or a monocyclic or bicyclic ring optionally containing N, O or S;

Y is an optionally substituted 5-12 membered heterocyclic ring containing a nitrogen atom, said heterocyclic ring may be monocyclic or fused, and is aromatic or partially aromatic;

A and R¹ are independently halo, CF₃, cyano, nitro, OR, SR, NR₂, COOR, CONR₂, NSO₂R, OSO₂R, or OSO₂NR, where each R is H, alkyl, alkenyl, alkynyl or aryl; or A and R¹ are independently an optionally substituted alkoxy (C₁₋₁₀), alkyl (C₁₋₁₀), alkenyl (C₂₋₁₀), alkynyl (C₂₋₁₀), aryl (5-12 members), arylalkyl, arylalkenyl, or arylalkynyl, each of which may optionally contain O, S, or N;

R² and R³ are independently H or an optionally substituted alkyl;

R⁴ is an optionally substituted heterocyclic ring or heteroaryl; or R⁴ comprises a urea, hydroxyurea, sulfamide, acetamide, guanidine, cyanamide, hydroxylamine, cyanamide, imidazolidine-2-one, or a nicotinamide moiety, each of which may be substituted with a heterocyclic ring;

R⁵ is H or alkyl;

1 and n are independently 0-4;

p is 0-1;

o and q are independently 1-4; and

r is 1-6.

In the above formula (7), at least one of R¹ and R² may not be H, and may be connected to form an additional ring such as an aryl or heteroaryl. In one example, two As may not form an additional ring. In another example, X is (CR³₂)ᵣ—R⁴, r is at least two, and R⁴ is 2-pyridinyl, quinolinyl, imidazolyl or furan.

In the above formula (7), X may be (CR³₂)ₒ—(CR³═CR³)ₚ—(CR³₂)_{q}—NR⁵₂, wherein each R³ and R⁵ are independently H and p may be zero. In particular embodiments, o and q together are 2-6. Alternatively, X may be (CR³₂)ᵣ—R⁴, wherein R⁴ is a heterocyclic ring or heteroaryl, each of which contains a nitrogen atom. For example, R⁴ may be azetidine, pyrrolidinyl, pyridinyl, thiophenyl, imidazolyl, or benzimidazolyl. Alternatively, X may be a monocyclic or bicyclic ring optionally containing N, O or S, such as cyclohexyl, piperidine, 8-aza-bicyclo[3.2.1]octane or 3-aza-bicyclo[3.2.1]octane. In yet another embodiment, X is an optionally substituted benzyl, particularly a disubstituted benzyl.

In the above formula (7), Y may be a 5-6 membered heterocyclic ring containing a nitrogen atom adjacent to the atom that is attached to the remainder of the molecule. The 5-6 membered heterocyclic ring may be fused to another ring. For example, Y may be pyridine, pyrimidine, pyrazine, indole, benzimidazole, benzothiazole, imidazole, isoquinoline, tetrahydroquinoline, pyridazine, thiazole, or benzoimidazole. In particular examples, Y is tetrahydroquinoline, particularly a 5,6,7,8 tetrahydroquinoline moiety, attached at position 8 to the remainder of the molecule.

In the above formula (7), each optionally substituted moiety may be substituted with a heteroatom, halo, CF₃, cyano, nitro, hydroxy, alkoxy, carbonyl, carboxy, amino, amido, imino, cyano, sulfonyl; C₁₋₆ alkyl or C₂₋₆ alkenyl each of which may contain N, O, or S; or substituted with aryl, heteroaryl, carbocyclic or heterocyclic ring, each of which may further be substituted with the same substituents.

Compounds having formula (7) and methods for synthesizing such compounds are set forth in WO 04/091518, which is incorporated herein by reference.

The CXCR4 antagonist may have formula (8)

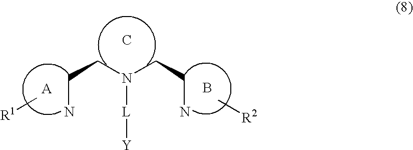

or the salts, prodrugs and stereoisomeric forms thereof,

wherein each of rings A and B is independently an optionally substituted 5-6 membered monocyclic heteroaryl;

ring C is an optionally substituted saturated or partially saturated 5-7 membered ring, and may contain a heteroatom in addition to nitrogen, wherein said heteroatom is N, O or S;

Y is H, a C₁₋₆ alkyl containing one or more heteroatoms, or a cyclic moiety, each of which is optionally substituted;

R¹ and R² are independently H, halo or an optionally substituted alkyl;

L is (CR³₂)₁ or NR(CR³₂)₁ wherein an alkyl bond may be replaced with an alkenyl or alkynyl bond;

1 is 1-6; and

each R³ is H or alkyl.

In the above formula (8), at least one of R¹ and R² may not be H when C is piperidinyl or 1,2,3,6-tetrahydropyridinyl and rings A and B are pyridinyl. In other embodiments, R¹ and R are not both naphthalenyl when ring C is piperidinyl and rings A and B are pyridinyl. In yet other embodiments, ring C is not 4-oxo-piperidine-3,5-dicarboxylic acid if L-Y is CH₃; and ring C is not 4-hydroxy-1,2,5,6-tetrahydro-pyridine-3-carboxylic acid ester if L-Y is benzyl.

In the above formula (8), R¹ and R² may be at positions adjacent the bonds to ring C. In one example, R¹ and R² are independently unsubstituted alkyl, such as methyl.

In the above formula (8), each of rings A and B may be pyridine, pyrimidine, pyrazine, pyridazine, 1,2,3-triazine, 1,2,4-triazine, 1,3,5-triazine, 1,2,4,5-tetrazine, pyrrole, imidazole, pyrazole, 1,2,3-triazole, 1,2,4-triazole, tetrazole, thiazole, oxazole, isothiazole, isoxazole, 1,2,3-thiadiazole, 1,3,4-thiadiazole, 1,2,3-oxadiazole, 1,3,4-oxadiazole, quinoline, isoquinoline, quinoxaline, quinazoline, phthalazine, cinnoline, 1,2,3-benzotriazine, 1,2,4-benzotriazine, indole, benzimidazole, 1H-indazole, benzoxazole, benzthiazole, benz[d]isoxazole, benz[d]isothiazole, or purine. In particular examples, each of rings A and B is pyridine, pyrimidine, imidazole, or benzimidazole, and each of rings A and B may be identical. Each of rings A and B may also contain a single substituent, which may be identical, at the position adjacent to the bond linking the rings to ring C.

In the above formula (8), ring C may be a saturated ring, or may contain a double bond. For example, ring C may be pyrrolidine, piperidine, hexahydro-1H-azepine, piperazine, morpholine, thiomorpholine, azepane, azocane, 2,3,4,7-tetrahydro-1H-azepine, 2,3,6,7-tetrahydro-1H-azepine, 3-pyrroline, 1,2,3,6-tetrahydropyridine, isoindoline, 1,2,3,4-tetrahydroisoquinoline, 2,3,4,5-tetrahydro-1H-benzo[d]azepine, 2,3,4,5-tetrahydro-1H-benzo[c]azepine, cyclobutane, cyclopentane, cyclohexane, cycloheptane, cyclooctane, cyclopentene, cyclohexene, cycloheptene, cyclooctene, tetrahydropyran, tetrahydrothiopyran, oxepane, thiepane, oxocane, or thiocane. In particular examples, ring C is pyrrolidine, piperidine, piperazine or hexahydro-1H-azapine. Ring C may be substituted with an optionally substituted alkyl, halo, cyano, oxime, OR or C═N—OR, wherein R is an optionally substituted alkyl.

In the above formula (8), Y may be selected from the group consisting of:

—(CR₂)ₘNR₂,

—(CR₂)ₘNR₂(CR₃),

—(CR₂)ₘNR(CR₂)ₘNR₂,

—(CR₂)ₘNR(CR₂)ₘNR(CR₂)ₘNR₂,

—(CR₂)ₘOR,

—(CR₂)ₘCO(CR₂)ₘOR,

—(CR₂)ₘCO(CR₂)ₘNR₂,

—(CR₂)ₘCO(CR₂)ₘNR(CR₂)ₘNR₂,

—(CR₂)ₘNRCO(CR₂)ₘNR₂,

—(CR₂)ₘNR(CR₂)ₘCO₂R,

—(CR₂)ₘNR(CR₂)ₘCOR,

—(CR₂)ₘNR(CR₂)ₘSO₂R,

—(CR₂)ₘNRCO(CR₂)ₘNR(CR₂)ₘNR₂,

—(CR₂)ₘNRCO(CR₂)ₘNR(CR₂)ₘNR(CR₂)ₘNR(CR₂)ₘNR₂,

—(CR₂)ₘNR(CR₂)ₘOR,

—(CR₂)ₘCR═NOH,

—(CR₂)ₘCONR(CR₂)ₘOR, —(CR₂)ₘN[(CR₂)ₘCO₂R₁₂,

—(CR₂)ₘ ONRCONR₂,

—(CR₂)ₘ-Z

—(CR₂)ₘNR—(CO)ₘZ,

—(CR₂)ₘNR—(CR₂)ₘZ, and

—(CR₂)ₘ—CR═N=Z;

wherein each R is H or an optionally substituted alkyl,

each m is independently 0-4; and

Z is an optionally substituted aromatic or heteroaromatic moiety containing 5-12 ring members.

In particular embodiments, Y is (CH₂)₁NR₂ and 1 is 1-10. Alternatively, Y may be a 5-12 membered aromatic, heteroaromatic, or a heterocyclic moiety, each of which may be a monocyclic or fused ring. For example, Y may be phenyl, imidazole, pyridine, thiophene, pyrrolidine, pyrazole, piperidine, azetidine, benzimidazole, benzo[d]isoxazole, or thiazole. Furthermore, Y may optionally be substituted with halo; cyano; nitro; alkoxy; halogenated alkyl; substituted carbonyl; a cyclic moiety such as a 5-12 membered aryl or heteroaryl containing N, O or S; or an alkyl, alkenyl, or a heteroalkyl moiety optionally containing one or more N, O, S, each of which is optionally substituted and optionally in the form of oxides. In particular examples, Y is substituted with pyridine, phenyl, piperidine or 2H-tetrazole.

In the above formula (8), each optionally substituted group may be substituted with inorganic moieties such as a heteroatom, halo, nitro, hydroxy, carboxy, amino, amido, cyano, or sulfonyl; or may be substituted with alkyl (C₁₋₁₀), alkenyl (C₂₋₁₀), alkynyl (C₂₋₁₀), aryl (5-12 members), arylalkyl, arylalkenyl, and arylalkynyl, each of which may optionally contain a heteroatom such as O, S, or N, and each of which may further be substituted with the same substituents. For example, each optionally substituted alkyl may be substituted with a heteroatom such as N, O, or S, or with a carbocyclic, heterocyclic, aryl or heteroaryl substituent.

Compounds having formula (8) and methods for synthesizing such compounds are set forth in WO 04/093817, and in U.S. patent application Ser. No. 10/977,221, filed 28 Oct. 2004, each of which is incorporated herein by reference.

1. A method to mobilize progenitor and/or stem cells from the bone marrow to the peripheral blood of a subject comprising administering to the subject an effective amount of at least one CXCR4 inhibitor or a pharmaceutically acceptable salt thereof in combination with an effective amount of at least one VLA-4 inhibitor or a pharmaceutically acceptable salt thereof.

2. The method of claim 1, further comprising harvesting the mobilized cells from the peripheral blood.

3. The method of claim 2, wherein the harvesting is by apheresis.

4. The method of claim 2, comprising culturing the harvested cells ex vivo.

5. The method of claim 2, further comprising administering the harvested cells to a recipient subject.

6. The method of claim 5, wherein the recipient subject is the same as the donor subject.

7. The method of claim 1, wherein the CXCR4 inhibitor is AMD3100 or a pharmaceutically acceptable salt thereof.

8. The method of any of claim 1, wherein the VLA-4 inhibitor is AMD15057 or a pharmaceutically acceptable salt thereof.

9. A method to enhance the effectiveness of a chemotherapeutic treatment or a radiotherapy in a subject afflicted with a hematopoietic or myeloid malignancy comprising administering to the subject an effective amount of at least one CXCR4 inhibitor or a pharmaceutically acceptable salt thereof in combination with an effective amount of at least one VLA-4 inhibitor or a pharmaceutically acceptable salt thereof.

10. The method of claim 9, wherein the malignancy is a lymphoma or leukemia.

11. The method of claim 9, wherein the CXCR4 inhibitor is AMD3100 or a pharmaceutically acceptable salt thereof.

12. The method of claim 9, wherein the VLA-4 inhibitor is AMD15057 or a pharmaceutically acceptable salt thereof.

13. The method of claim 9, wherein the CXCR2 agonist is GROβ protein.

14. A method to treat multiple myeloma in a subject comprising administering to the subject an effective amount of at least one CXCR4 inhibitor or a pharmaceutically acceptable salt thereof in combination with an effective amount of at least one VLA-4 inhibitor or a pharmaceutically acceptable salt thereof.

15. The method of claim 14, wherein the CXCR4 inhibitor is AMD3100 or a pharmaceutically acceptable salt thereof.

16. The method of claim 14, wherein the VLA-4 inhibitor is AMD15057 or a pharmaceutically acceptable salt thereof.

17. A pharmaceutical composition comprising as active ingredients at least one CXCR4 inhibitor or a pharmaceutically acceptable salt thereof and at least one VLA-4 inhibitor or a pharmaceutically acceptable salt thereof and a pharmaceutically acceptable excipient.

18. The method of claim 1, wherein the CXCR4 inhibitor is AMD3100 or a pharmaceutically acceptable salt thereof and the VLA-4 inhibitor is AMD15057 or a pharmaceutically acceptable salt thereof.

19. The method of claim 1, wherein the CXCR4 inhibitor is AMD3100 and the VLA-4 inhibitor is AMD15057.

20. The method of claim 1, wherein the CXCR4 inhibitor is AMD3465 or a pharmaceutically acceptable salt thereof.

21. The method of claim 1, further comprising administering G-CSF to the subject.

22. The method of claim 9, wherein the CXCR4 inhibitor is AMD3100 or a pharmaceutically acceptable salt thereof and the VLA-4 inhibitor is AMD15057 or a pharmaceutically acceptable salt thereof.

23. The method of claim 9, wherein the CXCR4 inhibitor is AMD3100 and the VLA-4 inhibitor is AMD15057.

24. The pharmaceutical composition of claim 17, wherein the CXCR4 inhibitor is AMD3100 or a pharmaceutically acceptable salt thereof and the VLA-4 inhibitor is AMD15057 or a pharmaceutically acceptable salt thereof.

25. The pharmaceutical composition of claim 17, wherein the CXCR4 inhibitor is AMD3100 and the VLA-4 inhibitor is AMD15057.
